(19) United States
(12) Patent Application Publication
(10) Pub. No.: US 2020/0211686 A1
(43) Pub. Date: Jul. 2, 2020

(54) METHOD FOR INTERFACING MEDICAL INFORMATION BETWEEN A MEDICAL INFORMATION EXCHANGE AND COMPUTING ENTITIES

(71) Applicant: Luc BESSETTE, Montreal (CA)

(72) Inventor: Luc BESSETTE, Montreal (CA)

(21) Appl. No.: 16/534,034

(22) Filed: Aug. 7, 2019

Related U.S. Application Data
(63) Continuation of application No. 14/916,785, filed on Mar. 4, 2016, now abandoned.
(60) Provisional application No. 61/887,709, filed on Oct. 7, 2013.
(60) Provisional application No. 61/893,528, filed on Oct. 21, 2013.
(60) Provisional application No. 62/031,518, filed on Jul. 31, 2014.
(60) Provisional application No. 61/874,671, filed on Sep. 6, 2013.

(51) Int. Cl. G16H 20/10; G16H 10/60; G06Q 10/00; G06Q 50/22

(52) U.S. Cl. CPC G16H 20/10; G06Q 50/22; G06Q 10/00; G16H 10/60

(57) ABSTRACT

Systems and methods for processing prescription information submitted to an electronic medical record system implemented in a data network including multiple nodes linked by communication paths is disclosed. The systems and methods disclosed include receiving at a server arrangement implemented at a first node a prescription issued from a second node associated with a doctor, this prescription conveying a particular brand name drug prescribed Laboratory B by the doctor to a patient, searching a database associating brand name drug names with corresponding chemical compound names, to identify a chemical compound name corresponding to the particular brand name drug, storing prescription information derived from the prescription in a particular record of the patient, and in response to a request for a pharmacy associated with a third node to access the prescription information in the patient record snaking the prescription information available to the third node.

### CROSS-REFERENCE TO RELATED APPLICATIONS

This application claims priority from U.S. Provisional Patent Application No. 61/874,671 filed on Sep. 6, 2013, U.S. Provisional Patent Application No. 61/887,709 filed on Oct. 7, 2013, U.S. Provisional Patent Application No. 61/893,528 filed on Oct. 21, 2013, U.S. Provisional Patent Application No. 62/031,518 filed on Jul. 31, 2014 and are hereby incorporated by reference herein.

### FIELD OF THE INVENTION

The present invention relates to the field of information distribution systems. More specifically, it pertains to a device and method for interfacing medical information between a Medical Information Exchange on a network and computing entities.

### BACKGROUND

Electronic Medical Record Systems (EMRS) are a collection of electronic medical information or health records for a group of patients of institutions such as hospitals or physicians' offices. Currently, EMRS are populated through local input of data at local institutions. Furthermore, the majority of EMRS are maintained at local institutions without any interconnectivity between different institutions.

A Medical Information Exchanges (MIE), also referred to as a summary multi-media medical record system, provides the capability of exchanging medical information or health records for patients between different institutions such as hospitals or physicians' offices. Currently, in Canada certain provinces are in the process of implementing MIEs on a regional basis. For example, the summary medical record system is called DSQ in Quebec.

### SUMMARY OF THE INVENTION

In accordance with a first aspect, the invention relates to a method for processing prescription information submitted to an electronic medical record system implemented in a data network including multiple nodes linked by communication paths, the multiple nodes including a first node, a second node, and a third node remote from each other. This method including receiving at a server arrangement implemented at the first node, a prescription issued from the second node associated with a doctor, this prescription conveying a particular brand name drug prescribed by the doctor to a patient. This method further including searching a database associating brand name drug names with corresponding chemical compound names, to identify a chemical compound name corresponding to the particular brand name drug. This method further including storing prescription information derived from the prescription in a medical record of the patient, the prescription including this chemical compound name identified as corresponding to the particular brand name drug. This method further including in response to a request from a pharmacy associated with the third node to access the prescription information in the medical record making the prescription information available to the third node including the chemical compound name. This method further including receiving at the server arrangement from the third node prescription fulfillment information indicating that the prescription has been fulfilled.

In accordance with a second aspect, the invention relates to a method for processing prescription information submitted to an electronic medical record system implemented in a data network including multiple nodes linked by communication paths, the multiple nodes including a first node, a second node, and a third node remote from each other. This method including receiving at a server arrangement implemented at the first node, a prescription issued from the second node associated with a doctor, this prescription conveying a particular brand name drug prescribed by the doctor to a patient. This method further including searching a database associating brand name drug names with corresponding chemical compound names, to identify a chemical compound corresponding to the particular brand name drug. This method further including storing prescription information derived from the prescription in a medical record of the patient. This method further including in response to a request from a pharmacy associated with the third node to access the prescription information in the medical record making the prescription information available to the third node. This method further including receiving at the server arrangement from the third node prescription fulfillment information indicating that the prescription has been fulfilled and that the chemical compound has been substituted for a different chemical compound.

Other aspects and features of the present invention will become apparent, to those ordinarily skilled in the art, upon review of the following description of the specific embodiments of the invention.

### BRIEF DESCRIPTION OF THE DRAWINGS

FIG. 1 is a block diagram of an example Medical Information Exchange (MIE), where the MIE is connected to various computing entities and electronic file management systems of hospitals and physicians' offices.

FIGS. 2A and 2B represent possible document layouts for a record on the MIE.

FIGS. 3A and 3B are flowcharts of possible processes for exchanging information on drug prescriptions between clients at a pharmacy and the MIE.

FIG. 4 represents a possible layout of the user interface as implemented at the computing entity in a pharmacy connected to the MIE.

FIG. 5 is a block diagram of the MIE architecture implemented in a data communication network in which embodiments of the invention may be implemented.

FIG. 6 represents a possible layout for the user interface of the computing entity at a physician's office, allowing the physician to prescribe drugs to a patient.

FIG. 7A is a block diagram illustrating an example of the structure of a database containing prescription drug information that may be part of the MIE.

FIG. 7B is a block diagram illustrating an example of the structure of a database containing prescriptions associated with patients that may be part of the MIE.

FIG. 8A is a flowchart for a possible process of looking up a chemical compound name for a brand name drug.

FIG. 8B is a flowchart for a possible process of removing a brand name drug from a prescription having a chemical compound name.

FIG. 9 illustrates the architecture of a prescription drug management system implemented at a pharmacy or a pharmacy chain, which interfaces with the computer system of an insurance provider to manage reimbursements to clients of the cost of prescription drugs, according to one embodiment of the invention.

FIGS. 10A and 10B illustrate possible table or data structure layouts in a database on a server.

FIG. 11 is a flowchart for a possible process for managing the substitution of brand name drugs by generic drugs.

FIG. 12 represents a possible user interface layout on the computing entity at a pharmacy for managing drug substitutions.

FIG. 13 is a continuation of the flowchart in FIG. 11 and illustrates the possible process of submitting a selection or substitution to an insurance provider.

FIG. 14 represents a possible user interface arrangement on the computing entity at a pharmacy for managing drug substitutions which also provides to the pharmacist the insurance provider's coverage information for each substitution.

FIG. 15 is an example of a MIE connected with computing entities at a physicians' offices and at pharmacies.

FIGS. 16A-16D are examples of patient records a specific point in time.

FIGS. 17A-17D are examples of the patient records of FIGS. 16A-16D at a later point in time.

FIGS. 18A-18D are examples of patient records of FIGS. 17A-17D at an even later point in time.

FIG. 19 is an example of a screen shot of a physician's computing entity.

FIGS. 20-28 are example screen shots of pharmacists' computing entities.

FIGS. 29A-29C are examples of patient records where the patients record does not contain brand name drug information.

FIG. 30 is an example of a screen shot of a pharmacist's computing entity.

The invention will now be described in greater detail with reference to the accompanying diagrams, in which:

### DETAILED DESCRIPTION OF THE PREFERRED EMBODIMENTS

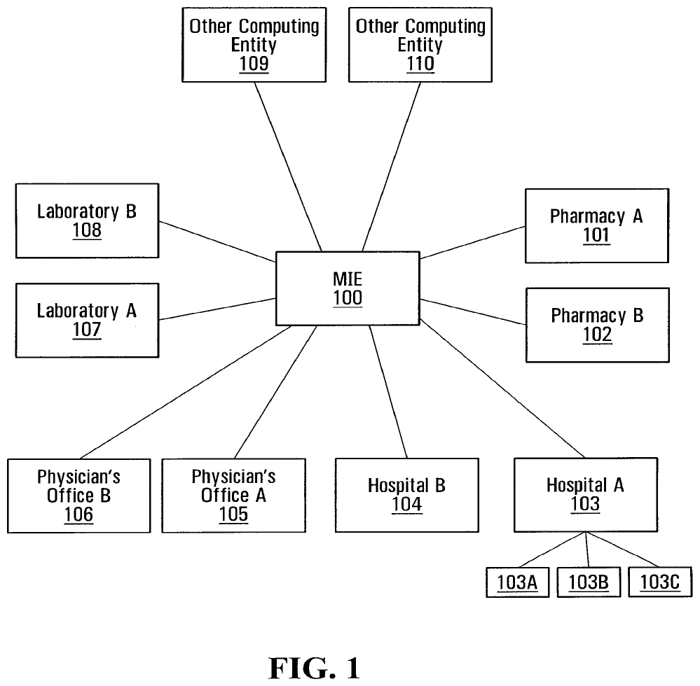

FIG. 1 illustrates a Medical Information Exchange (MIE) 100, which may also be referred to as an electronic medical record system, where the MIE 100 is connected to various computing entities and/or electronic file management systems 101 to 110.

### Medical Information Exchange (MIE)

The MIE 100 may be capable of exchanging patients' medical records, health records or other medical or health information (herein “medical records”), for example, between different institutions such as a hospital 103, a physician's office 106, a laboratory 107, a pharmacy 101, or some other computing entity 109. The MIE may consist of a server arrangement including one or multiple servers that store electronic medical records or electronic summary medical records. The medical records stored on the MIE may be stored in a single location on the MIE or may be stored in a distributed fashion throughout the data communication network, which may include servers, disk-arrays, computers or other nodes connected to each other by data communication links. In other words, the MIE may be implemented in a distributed nature in a data network including multiple nodes linked by communication paths. That is, the MIE may be implemented by one or more nodes in a data network.

In some embodiments, the MIE 100 may be implemented by at least one server having one or more databases stored in a computer readable memory, where the at least one server also having at least one computing unit or processor which is programmed with software, which when executed executes a number of steps as set out in the software.

The implementation of an MIE is known in the art; for example, see Canadian Patent No. 2,223,794 C or Canadian Patent Application No. 2,329,598 A1 both of which are incorporated herein by reference. As such, the MIE does not need to be described in detail because such systems are well within the reach of a person skilled in the art. Furthermore, for the purpose of the following description, a MIE may be known as a summary medical record system such as, for example, the one called DSQ in Quebec. In other words, the MIE may be electronic medical record system managed by the government or a government agency (e.g., regulatory agency, regulatory authority, regulatory body or regulator, public authority, government body, etc.).

In a specific example, a patient's medical records stored on the MIE may include information such as: prescribed medication, delivered medication, laboratory results, pathology reports, consultation reports, imaging reports and images themselves, ECG reports or the images themselves, surgical or procedure reports with or without images, allergies or medication intolerances, hospitalization summaries, physician summaries, etc. The information stored in a patient's medical record is not limited to the non-exhaustive list given above, a person skilled in the art would understand that other types of patient and medical information may also be stored in a patient's medical record on the MIE.

More specifically, the patient's medical records stored on the MIE be of a summary nature and include information such as a summaries of: Administrative Data, Permanent Biological Data, Significant Antecedents, Current Medical Conditions, Biological Data, Prescribed and/or Delivered Medications, Laboratory Results, Pathology Reports, Consultation Reports, Imaging Reports and Images, ECG reports and/or ECG Images, Surgical or Procedure Reports, Allergies and/or Medication Intolerances, Hospitalization Summaries or Physician Summaries. Furthermore, each summary may include a pointer which points to more complete information regarding each summary. It is appreciated that such a configuration may allow information stored in each summary record to be of a distributed fashion where information may be store in different or multiple nodes of a data network. For example, the ECG reports summary may list pointers to where the ECG images are actually stored. Similarly, different laboratory reports, images, prescribed prescriptions, and so forth, may be at different nodes of the data network and the summary records contains points that point to the different nodes in the network that store the related information.

By way of example, a patient may have a record stored on the MIE where the data for different parts of the record are not stored directly on the MIE. For instance, a MIE may list prescribed medications and also store the details of each prescription locally but list, for example, imaging reports and points to a record in a laboratory's electronic record system where the images themselves are stored. It could also be possible for the MIE to list the prescribed medication and point to the physician's office where the details of each prescription are stored. The invention is not limited to this example, a person skilled in the art would clearly understand that any number of combinations of different types of records where some records are stored on the MIE and others are stored remotely on other electronic record systems could exist. Similarly, it could be possible for the MIE to not contain any data records other than a list of records where the data for each record is stored in other electronic record systems, and where each element in the list of records points to the location of where the data record is stored.

Furthermore, it may also be possible for all of a patient's data for his or her record to be stored on the MIE. For example, a patient's prescribed medication, delivered medication, laboratory results, pathology reports, consultation reports, imaging reports and images themselves, ECG reports or the images themselves, surgical or procedure reports with or without images, allergies or medication intolerances, hospitalization summaries, physician summaries, may all be stored on the MIE.

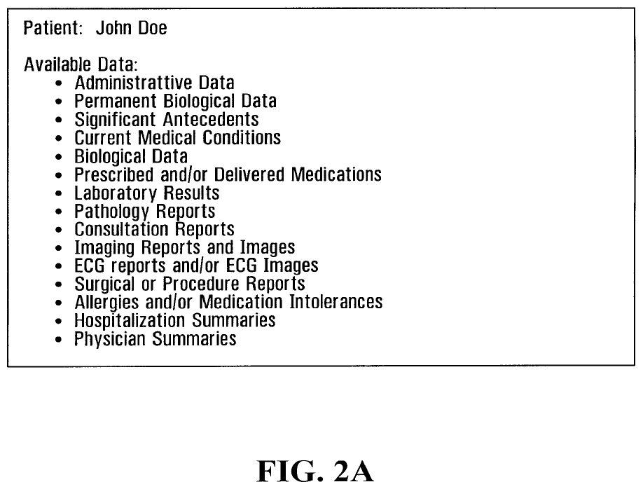

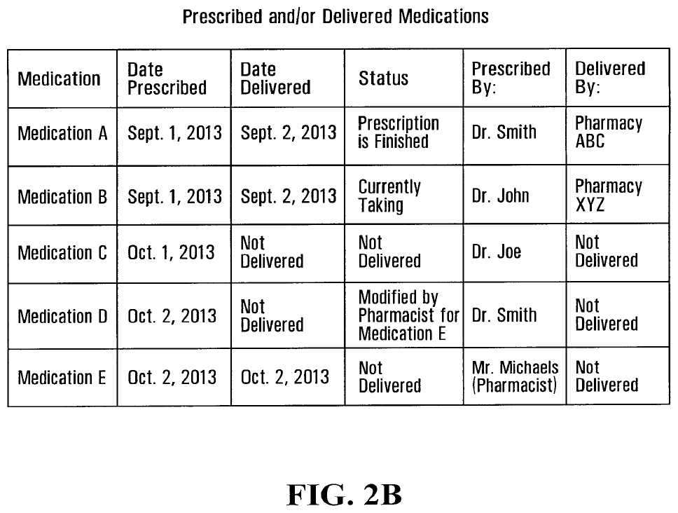

FIG. 2A is an example of a user interface that lists information items stored in an electronic patient record stored in the MIE. The patient record lists medical information categories in which patient specific information is stored. For example, FIG. 2B shows a category for “Prescribed and/or Delivered Medications”. A person skilled in the art would understand that there are many different ways to arrange the data in a medial record and that the present invention is not limited to this illustrated arrangement.

### Patient Authorization

For the MIE to store a patient's electronic record, a patient may be required to give authorization or consent. The consent may indicate that parties such as doctors, lab technicians, pharmacists, etc, are also able to access a patient's medical record. In this consent the patient may be able to specify certain periods of time during which the patient withdraws the consent. For example, if the patient was in a psychiatric facility the patient may want to exclude this event from the record on the MIE.

It may be possible for a patient to give electronic authorization to the MIE to store or share their medical record. For instance, a patient may authorize himself to the MIE by the use of biometrics and/or a smart phone or other mobile computing device. Also, it may be desirable for a patient to send electronic authorization of their medical record to a doctor or pharmacist in a different country or province/state, which would not normally have access to the MIE or the patient's medical record.

### Computing Entity

Referring to FIG. 1, a computing entity (any one of 101 to 110) may be a computer, a workstation, or a portable or mobile computing device. The computing entity may be connected to an internet connection or other networks (e.g., local area network (LAN), or a wide area network (WAN)). Furthermore, the computing entity is connected to the MIE which may be connected via a LAN, or a WAN (either of which a LAN or WAN may be considered part of an intranet or internal network). The computing entity may also be connected to the MIE through an internet connection (i.e., not part of an intranet or internal network) provided by an internet service provider or other service provider. The computing entities connection to internet, intranet or internal network may be a wired or wireless connection. LANs, WANs, intranets, internal networks, and internet connections do not need to be described in detail because such networks and connections are well known to a person skilled in the art. It is appreciated that the computing entity may be referred to as a node which is part of the data network that may including multiple nodes linked by communication paths.

In other words, a computing entity is a computer platform which includes a CPU executing software and acting as a node in the data network, capable to exchange data with other nodes in the data network over communication links that may be wire line based or wireless. A computing entity may be a mobile device or it may be stationary.

In some embodiments, the computing entity may be implemented by at least one portable or non-portable computer having one or more databases stored in a computer readable memory, where the at least one portable or non-portable computer also having at least one computing unit or processor which is programmed with software, which when executed executes a number of steps as set out in the software.

In FIG. 1 the computing entities 101 and 102 are illustrated as computers at pharmacies, computing entities 103 and 104 are illustrated as computers at hospitals, computing entities 105 and 106 are illustrated as computers at physicians' offices, and computing entities 107 and 108 are illustrated as computers at laboratories. However, other computing entities 109 or 110 could exist. Such other computing entities could be any type of computing device of physicians, nurse practitioners, physician assistants, dentists, optometrists, radiologists, druggists, pharmacist assistants, etc. Furthermore, Hospital A 103 is illustrated in FIG. 1 as a server or computing entity connected to the MIE, where the Hospital A 103 consists of multiple computers or computing entities 103a, 103b, 103c, as part of a network. These multiple computers or computing entities 103a, 103b, 103c may be part of a network that communicates with a server 103 that is connected to the MIE. Alternatively, these multiple computers or computing entities 103a, 103b, 103c may be connected directly to the MIE (not illustrated in FIG. 1). Furthermore, any of computing entities 101 to 110 may contain multiple computing entities or computers (not illustrated in FIG. 1 for 101-102, 104-110) all of which may be connected to the MIE either directly or through an intermediary (such as a server). Any of the multiple computing entities may have electronic file management systems which may be shared with other computing entities within the multiple computing entities network.

The computing entities 101 and 102 residing in a pharmacy may contain pharmacy electronic record system software and/or drug interaction software. The pharmacy electronic record system software and the drug interaction software may be part of the same software package or integrated into a single piece of software. The pharmacy electronic record system software and/or drug interaction software may store or access information locally on the computing entity or remotely on storage devices, other computers or servers within an internal network or external network (which may be accessed through an internet connection). The pharmacy electronic record system software and/or drug interaction software is herein referred to as “pharmacy software”.

The invention is not limited to the number of computing entities or the configuration of computing entities illustrated in FIG. 1. A person skilled in the art would understand that there may be numerous computing entities, configured in many different possible arrangements.

### Prescription

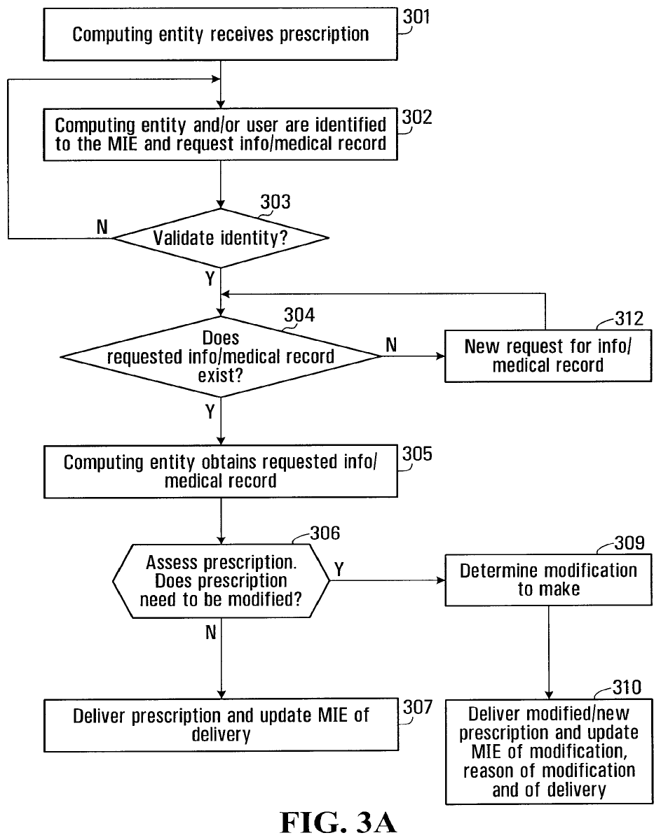

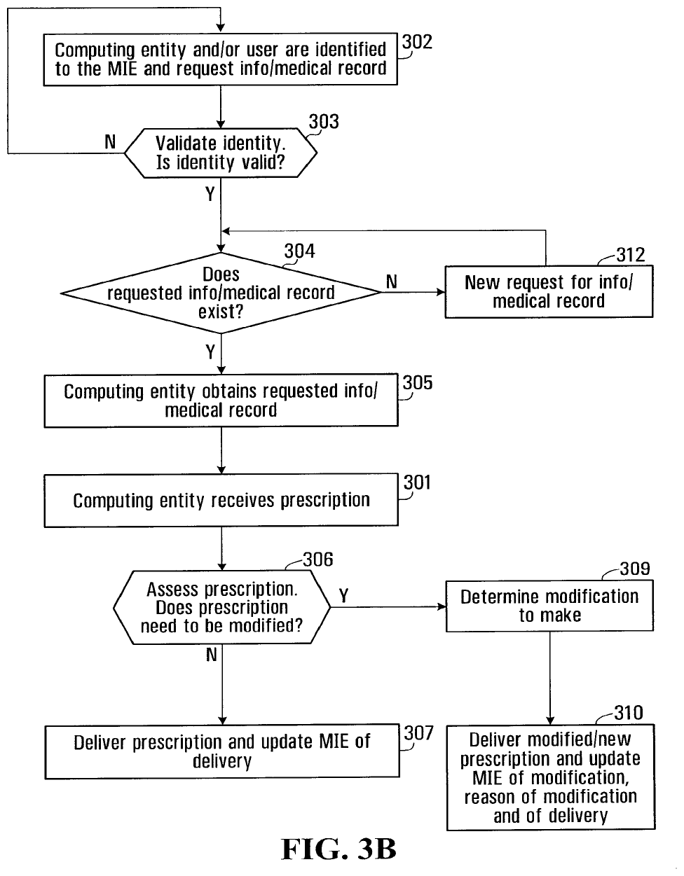

FIG. 3A illustrates a possible process for managing prescription drug information. At step 301, a pharmacist at the computing entity 101 receives a prescription from a patient or individual. The patient or individual may, for example, receive the prescription from a doctor, optometrist, dentist, nurse practitioner, or pharmacist. The prescription associated with an individual may include the patients name, the prescribed drug or medication, and the dosage (which may include the frequency and length of time to take the prescribed medication), etc. For example, the prescription may be handwritten by a doctor or other qualified professional which may be later entered in to an EMRS (or MIE), created by a doctor or other qualified professional as an electronically prescribed prescription. The electronically prescribed prescription may be created in a local EMRS (which may be later entered into a MIE), or created in a local EMRS which is connected to the MIE (which updates the MIE at the time of creating the prescription), or created directly into the MIE. The patient or individual may take the handwritten or a printed out copy of an electronic prescription to a pharmacy where a user, such as a pharmacist or assistant, enters or inputs the received prescription into the pharmacy software on the computing entity.

In the case where the electronic prescription is registered with the MIE via a computing entity associated with the doctor, the identity of the doctor may be validated by the MIE prior to registration of the prescription. For example, the doctor may log in to the MIE by providing a user name and password, which may also include the use of a hardware token.

Upon electronically prescribing the medication, the MIE may register this prescription. One implementation of this invention would be also to geolocate the drug store which is the closest either to the work location or the residing address of the patient and send from the doctor's office an electronic transmission of the prescription to the computing entity of this facility. This electronic transmission of the prescription could be a transmission sent via the medical information exchange. Similarly, the prescription may be sent via an electronic transmission from the doctor's office to the computing entity of any facility of the patients or doctors choosing. This facility would then receive and prepare the prescription for the patient and deliver it according to preferred instructions.

### Identification

As illustrated in FIG. 3A at step 302, the pharmacist or user identifies himself (or herself) to the MIE 100 through his (or her) computing entity 101 and the pharmacy software. For instance, the pharmacist may have a password, an authentication key, an USB key device plugged into the computing entity, or other portable electronic device to identify himself to the MIE. Other forms of identification may include the use of a smart-card and smart-card reader connected to the computing device, or a biometric reader connected to the computing device which may take a biometric reading of the pharmacist. Many different identification techniques are known to the person skilled in the art, and the present invention is not limited by these aforementioned examples.

The user may also use a combination of a USB key device and a password to identify himself to the MIE. For instance, the USB key device may be plugged into a USB port on the computing entity 101. Alternatively, the USB key may be plugged into a port on a hub/centralizer which connects to a server an acts as a gateway to allow access to the MIE on the computing entity 101 for the entry of a password. Once the USB key is plugged in, then the user can be prompted to provide his password to access the MIE on the computing entity 101.

### Validation

Upon identifying himself to the MIE 100, the MIE 100 validates the pharmacist's (computing entity's 101) current authorization to have access, as illustrated in FIG. 3A step 303. The security processes of identifying and validation between a computing entity and network or server such as the MIE are well known to the person skilled in the art, and as such are not discussed in detail. Concurrent or subsequent to the validation the MIE may also register all of the pharmacist's ulterior requests for information and validate that the patient has data and has provided authorization to provide this data to a third party. Upon validation, the information is retrieved and input is made in the pharmacy software on the computing entity.

### Request of Information from the MIE

A request for information from the MIE may take place at the same time the computing entity or the user of the computing entity identifies himself to the MIE. Although in FIG. 3A, the step of identification and request for information takes place at the same step (step 302), alternatively the request for information from the MIE may take place after the computing entity or user of the computing entity has identified himself and his identify has been validated (i.e., after step 303). Upon a request for information, such as a medical record for the patient associated with a prescription, and if a medical record for the patient exists and the patient has previously given authorization (step 304), the patient's medical record is obtained (step 305), and is then visible on the screen of the pharmacy software on the computing entity. Furthermore, in the case where there are multiple computing entities part of a network of a pharmacy, it may be possible for the record obtained from one computing entity to be accessed by any of the other computing entities in the network.

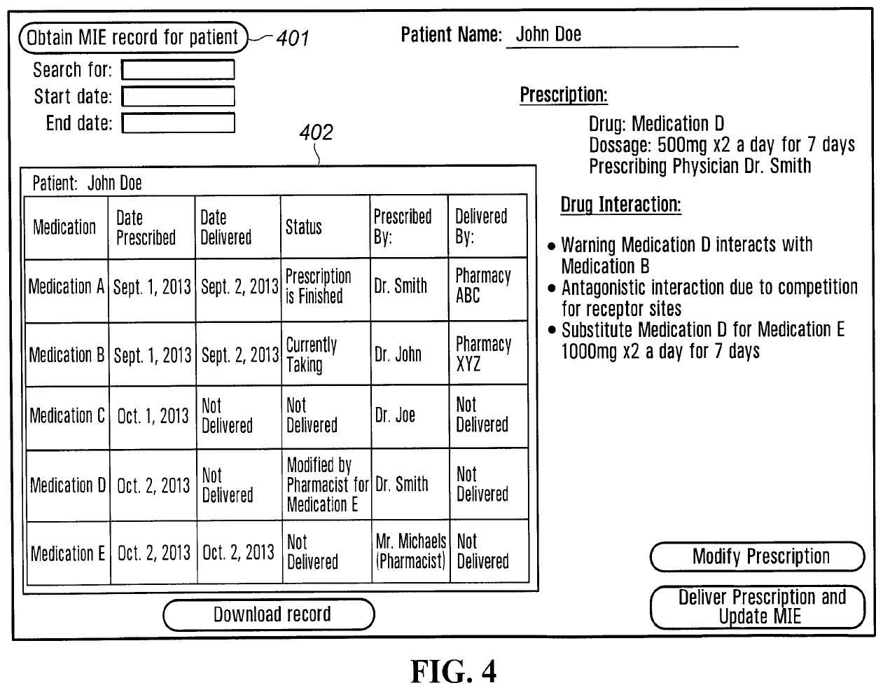

Referring now to FIG. 4, for example, half of a screen of the pharmacy software running on the computing entity may show the patient's record and the other half may show the pharmacist's record including the drug interaction results. The interfacing of the information provided via the MIE and the computing entity running the pharmacy software may be through the equivalent of a button 401 in the pharmacy software. Via clicking this button (or any other method of connection), data registered on the MIE may appear either via a specific window 402 or any other display mode so that the user of the computing entity may be made aware of the information collected in the MIE. The data displayed in the window 402 may include the data shown in FIG. 2A or FIG. 2b. Many other arrangements of the screen are possible, and a person skilled in the art would understand that the invention would not be limited to these configurations. Furthermore, this process of requesting information may only happen if the user has been validated (e.g., USB key and password) and if the patient has also allowed the MIE to proceed by having provided consent.

Then the pharmacist or user may also have the ability to query information available or even look for specific information by providing for example a first date and a second date, defining the time period looked for by the query of information done on the MIE (e.g. which are the drugs prescribed for this patient between Apr. 1, 2013 and now? or which are the laboratory results available between May 15, 2013 and now?).

Upon display of the information, the pharmacist or user may also have the opportunity to identify certain elements of the list displayed either by clicking on them or using any keyboard key or vocal instruction or touch screen command, to import these list's elements so that they will now be part of the database of the computing entity and pharmacy software for future reference. That is, the pharmacist may even download the patient's record (or parts of) to their computing entity (which may include any local network or external network storage devices). Note that if the patient changes their authorization to certain records stored on the MIE, this information may still be saved locally on the pharmacist's computing entity. Furthermore, the record stored locally on the pharmacist's computing entity may only contain information up to the time of retrieval. In other words, any updates done to the MIE for a patient's record after being obtained by the pharmacist's computing entity may not be visible unless the pharmacist requests a refresh/update from the MIE.

Alternatively to FIG. 3A, a prescription can be received according to FIG. 3B. In FIG. 3B the steps 302 to 305 and 306 to 310 are the same as in FIG. 3A. The main difference between FIG. 3A and FIG. 3B is that the prescription is received after accessing the patient's medical record. That is, step 301 occurs after step 305. According to FIG. 3B, a pharmacist may receive a prescription at the computing entity by obtaining or viewing a medical record stored in the medical information exchange and selecting a prescription to be received. For example FIG. 2B illustrates a prescription “Medication C” which is not yet delivered. The pharmacist could then select the prescription “Medication C” to be received at the computing entity and then deliver it by providing to the patient the drugs in the prescription.

### Prescription Delivery and Modification

The pharmacist may now verify the validity of the current medication. For example, the verification of the prescription may be done by looking at the prescription in window 402, to confirm it has been prescribed by a doctor and not yet delivered. The pharmacist may now look for any prescribed but not yet delivered medication. For example, the pharmacist may look at the prescriptions listed in window 402, which may contain the information as shown in FIG. 2B, to see if there are any prescriptions not yet delivered. Furthermore, a pharmacist may deliver a drug prescription already registered in the MIE but not previously delivered, regardless of whether or not the patient physically provides the pharmacist with a paper copy of the prescription.

The pharmacist may even adjust the prescribed medication if necessary or even substitute it if required or prescribe a new medication. For example, a pharmacist may look at a results of window which may show analysis results or drug information from analyzing the prescribed drug with the data in the patient's medical record. At step 306 of FIG. 3A or 3B, the computing entity then assesses the prescription to determine whether the prescription should be modified or not. In assessing whether a prescription should be modified, the computing entity may obtain drug information associated with the prescription. For instance, the drug information may be provided by drug interaction software. For example, drug interaction software may analyze a patient's existing medications with the prescribed drug to determine if a situation exists that affects the activity of either the prescribed drug or the current medications when both are administered together. Furthermore, drug interaction software may analyze other interactions such as interactions between drugs and food or natural supplements. Also, the drug information associated with the prescription could be information whether a less costly generic brand is available, or information if the pharmacy is out of stock of the prescribed drug.

More specifically, the pharmacist may import or receive a prescription in his pharmacy software on the computing entity and either delivers it as such, or:

1. Modify it after having assessed through input from the medical information exchange and the local drug interaction software that a modification is needed either in reason of:
Synergetic or antagonistic interaction with another drug which may be due to:
   competition for receptor sites; or
   increased affinity of a drug to a receptor site which prevents the other drug of binding as well its receptor and producing its pharmacologic effect; or
Action on two different receptors which produce an undesirable effect; or
Enzymatic inhibition or induction of cytochrome activity in the bowel or in the liver; or
Conflicting signal transduction mechanisms; or
Antagonic physiological systems activation; or
Absorption interactions caused by changes in motility caused either by modification of gastric pH, decreased drug solubility and formation of non-absorbable complexes with another drug in the intestinal lumen; or
Increased excretion of a drug either by kidney or the liver.

2. Modify it after having assessed through input from the medical information exchange and the local drug interaction software that the dosage is wrong or does not fit with either kidney or liver failure as evidenced by laboratory results available on the medical information exchange.

3. Modify it for a less costly generic brand if it is required by the insurer or the patient for economic reason.

4. Modify it after having assessed through input from the medical information exchange and the local drug interaction software that genetic markers present show that the patient is a fast or slow metabolizer of the specific drug prescribed to the patient as evidenced by the presence of some specific alleles.

5. Modify it if the medication is out of stock.

6. Modify the dosage or a medication if after having assessed through input from the medical information exchange and the local drug interaction software that a natural supplement the patient is taking adversely interacts with the medication either through:
Synergetic or antagonistic interaction with another drug which may be due to:
   competition for receptor sites; or
   increased affinity of a drug to a receptor site which prevents the other drug of binding as well its receptor and producing its pharmacologic effect; or
Action on two different receptors which produce an undesirable effect; or
Enzymatic inhibition or induction of cytochrome activity in the bowel or in the liver; or
Conflicting signal transduction mechanisms; or
Antagonic physiological systems activation; or
Absorption interactions caused by changes in motility caused either by modification of gastric pH, decreased drug solubility and formation of non-absorbable complexes with another drug in the intestinal lumen; or
Increased excretion of a drug either by kidney or the liver; or
epigenetic action on the expression or non expression of some genes.

7. Modify the dosage or a medication after having assessed through input from the medical information exchange and the local drug interaction software that a special diet adversely interacts with the medication either by:
Synergetic or antagonistic interaction with another drug which may be due to:
   competition for receptor sites; or
   increased affinity of a drug to a receptor site which prevents the other drug of binding as well its receptor and producing its pharmacologic effect; or
Action on two different receptors which produce an undesirable effect; or
Enzymatic inhibition or induction of cytochrome activity in the bowel or in the liver; or
Conflicting signal transduction mechanisms; or
Antagonic physiological systems activation; or
Absorption interactions caused by changes in motility caused either by modification of gastric pH, decreased drug solubility and formation of non-absorbable complexes with another drug in the intestinal lumen; or
Increased excretion of a drug either by kidney or the liver; or
Epigenetic action on the expression or non expression of some genes.

8. Modify the dosage or the medication after having assessed through input from the medical information exchange and the local drug interaction software that the individual's specific gut flora (microbiota) adversely interacts with the medication

9. Modify the dosage or the medication if after having assessed through input from the medical information exchange and the local drug interaction software that a metabolic substrate necessary for the required therapeutic effect of the medication or the avoidance of a significant side effect is deficient.

10. Any other suitable modification.

For example, the above modification may be determined at step 309 of FIG. 3A or 3B.

### Updating the MIE

Whenever, this verification or adjustment of a prescription is done, the pharmacy software and the computing entity may export the new revised data to the MIE which is now updated. In other words, the pharmacist enters via the user interface at the computing entity in the pharmacy the new prescription drug information or modified prescription drug information and that information is exported to the patient record in the MIE and permanently stored therein. Alternatively, the MIE may be updated when the prescription is delivered and paid for by the individual or patient. In the same time, it may process the request of payment to be sent to either the public or private insurer so that the suitable honorarium is paid. For example, when the pharmacist verifies and/or assesses the prescription and no modification is required, then the MIE is updated as illustrated in FIGS. 3A and 3B at step 307 which may occur at the time of assessment or at the time of delivery. Alternatively, when a pharmacist does modify the prescription then the MIE is updated as illustrated in FIGS. 3A and 3B at step 310. Furthermore, in other embodiments, the MIE may only be updated by the pharmacists once a day (e.g., at the end of the day) or when the pharmacists explicitly submits an update (e.g., clicking a button that says “update”).

After having made a modification or adjustment to a prescription, delivery of the medication, and billing the proper insurer either for the full amount or the amount minus the copay, the local pharmacy software may reciprocally feedback and update the MIE by:
providing the list of the new medication prescribed;
identifying any discovered drug interaction and/or its mechanism;
identifying significant kidney or liver failure that has to be taken in account in the prescription of drugs;
identifying any genetic marker that should be taken into account for proper dosage;
identifying the natural supplement which interacts with the medication;
identifying the elements of the special diet which interacts with the medication;
identifying the specific gut flora elements that interacts with the medication;
identifying the deficient metabolic substrates that cause inadequate therapeutic effect or side effect; or
etc.

### The Embodiments of the Invention are not Limited to Pharmacies

Although the embodiments discussed above is in relation to a pharmacist and pharmacy software, in accordance with another embodiment of the invention, the MIE is capable of exchanging medical information with physicians, nurse practitioners, physician assistants, dentists, optometrists, radiologists, druggists, pharmacist assistants, and the software that these individuals run on their computers or computing entities.

### Consultation of the MIE Prior to Prescribing a Prescription

In some other cases, the physician, the pharmacist, the nurse practitioner or the physician assistant may prescribe a new medication after having consulted the medical information exchange. Such prescription may be made with the awareness of any previous prescribed medication, medical antecedent as well as drug allergy. Upon electronically prescribing the medication, the MIE will register this prescription.

### Submission of Prescription to Pharmacy & Patient Notification

One implementation of this invention would be also to geolocate the drug store which is the closest either to the work location or the residing address of the patient and send the prescription to this facility which could prepare the medication for the patient and deliver it according to preferred instructions. A notice of the availability of the prescription as well as a further notice of the fact that it is already prepared and available at the drug store could be sent to the patient via e-mail or as a text message on his phone or his preferred digital assistant (for example i-watch). To secure the fact that the holder of the digital assistant is effectively the right person to notify, the computing entity or local electronic record system would previously require an electronic token identifying uniquely the end user to which this information is conveyed.

The same process would allow conveying information about laboratory results or any significant information to a specific patient who identifies himself to the gateway of the information system that has just received the information. Hence, the notice of new information may be pushed to a patient as soon as new data is available. However, information regarding the content of the data would not be processed without a confirmation of the identity of the said patient. Conversely, the patient himself may initiate a pull of any new information available after having identified himself to the system.

### Mining of the Databases

The ability of populating the MIE databases by the cooperative aggregation of significant added-value data via local medical or pharmaceutical records may prove instrumental in acquiring critical information in the fields of functional medicine, pharmacogenomics, nutraceutics, metabolic influence of the microbiota as well as environmental medicine. These databanks could be mined locally or on the medical information exchange by search engines. Such researches may prove in the fields listed.

If, moreover, one adds intelligent agents to the database that may automatically correlate influences and report significant statistical deviation, this rekindled medical awareness may support outstanding medical research and development that may greatly enhance our ability to treat human illnesses.

### Further Embodiments of the Invention

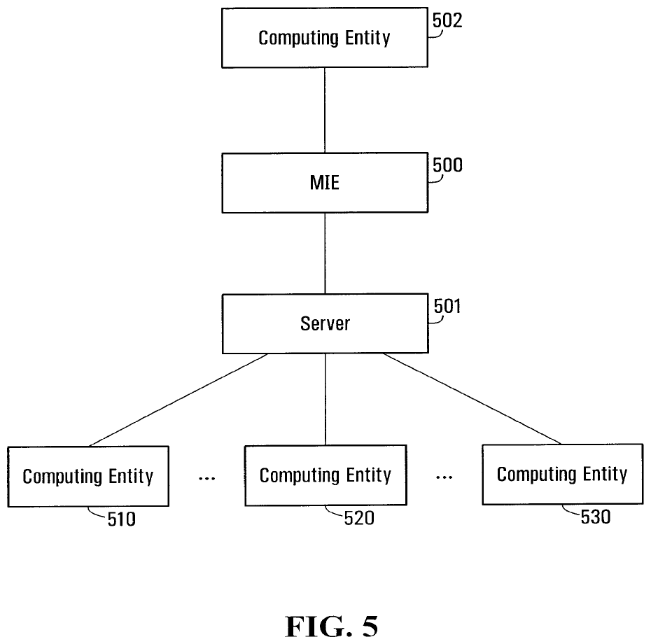

In another embodiment of the invention FIG. 5 illustrates a MIE 500 connected to a computing entity 502. The computing entity 502 may be located at a physician's office, or other professional's office that may provide prescriptions. The MIE is also connected to a server 501. The server 501 is connected to multiple computing entities 510, 520, and 530. The server 501 may be a server located at pharmacy, a server managed by a pharmacy chain, or a server managed by another service provider. The computing entities 510, 520, 530 may be located a pharmacy or drug store.

Although not illustrated in FIG. 5, the computing entity 502 could comprise one or more servers and a plurality of computing entities connected to the server in order to connect to the MIE 500. Furthermore, the illustration in FIG. 5 only shows a single server 501 and a single computing entity 502 at a physician's office, however, the present invention is not limited to such a configuration as a person skilled in the art would clearly understand that there may be multiple computing entities and multiple servers connected to the MIE 500.

The MIE 500 in this embodiment may be the same and/or configured in the same manner as the MIE 100 in the previous embodiment. Similarly, the computing entities 502, 510, 520, 530 may be the same and/or configured in the same manner as the computing entities 101, 102, 103A, 103B, 103C, 104, 105, 106, 107, 108, 109, 110.

### Computing Entity at Physician's Office

A physician at computing entity 502 may prescribe a prescription for a patient. The physician's computing entity may be running its own local EMRS or other software which may run locally to connect to or communicate with the MIE. Alternatively the physician's computing entity may be directly connected to MIE though software running on the physician's computer. For example, this could be by using a web-browser or other software that makes request and submit data to the MIE, and where all of the processing occurs at the MIE. The physician's computing entity may have a monitor or screen (which may include a touch screen) where the EMRS or other software has a user interface (UI) that the physician can use to interact with the EMRS or other software and in order to communicate with the MIE.

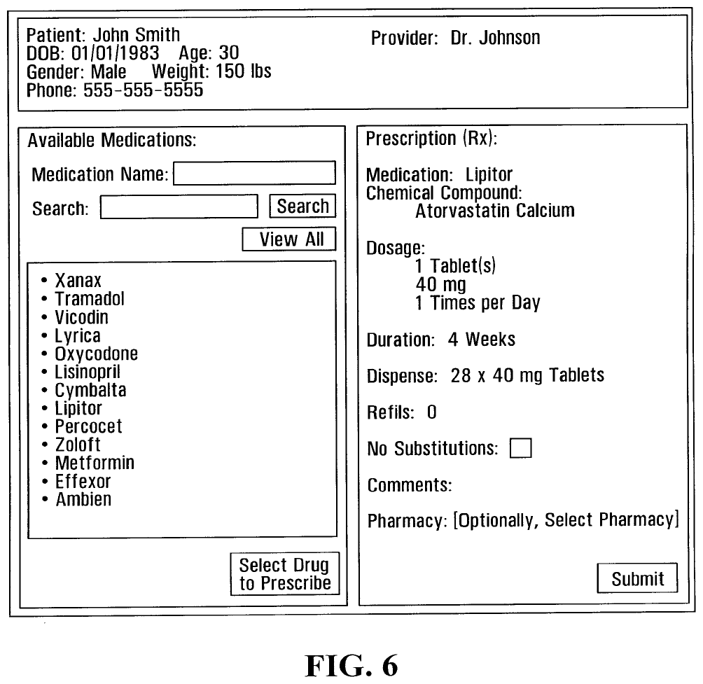

Referring now to FIG. 6, the physician enters in the drug to be prescribed into the UI. This may be in the form of the physician typing in the brand name. Alternatively, the physician may search for a drug listed in a local database or on a database on the MIE, the physician then may also view a list of all available drugs and then select a drug to prescribe. The drug to be prescribed may be in the form of brand name, generic name, or may be in the form of a chemical compound name or even a DIN (drug identification number) or any other equivalent drug identifier. In the case that the physician enters in or selects a brand name drug, the UI may dynamically generate the chemical compound for the brand name drug. That is, as the physician enters the brand name drug into the UI and the chemical compound automatically pops up or is visible in the UI. The UI may interact with the MIE to get the chemical compound name associated with the brand name drug or it may be dynamically generated by the software running local on the physician's computing entity. The physician may then select a dosage, a duration, the dispensing, the number of refills (if any), and determine whether substitutions are allowed or not. By way of example, there may be a check box that the physician may select if he does not want substitutions to be made at the pharmacy. Once the prescription is complete the physician then submits the prescriptions to the MIE. The submission may be in the form of a button which the physician clicks on or touches on the screen. The electronic prescription is then stored on the MIE. When the physician submits the prescription to the MIE, the physician may also select a pharmacy to also send the prescription to.

Alternatively, and not illustrated in FIG. 6, the chemical compound name may not be visible to the physician till after the physician submits the prescription to the MIE, the software executing on the MIE server which interacts with the physician's computing entity searches a database mapping drug brand names with corresponding chemical compound names, and either substitutes the entered brand name to the corresponding chemical compound name or adds the chemical compound name to the brand name. The MIE software may interact with the physician's computing entity to validate that the chemical compound is correct. By way of example, this validation may be in the form of a pop-up box which pops up after the physician clicks submit which list the chemical compound identified and asks the physician if this is correct or not.

As noted above, the UI may interact with the MIE to get the chemical compound name associated with the brand name drug or it may be dynamically generated by the software running local on the physician's computing entity. When the UI obtains the chemical compound for a brand name drug from the software running locally on the physician's computing entity this may include accessing servers within the physician's network to access a database that contains a listing of the chemical compound name for each brand name drug.

Although the above embodiment is illustrated by use of a physician the above mentioned computing entity and process may take place at any professional's computing entity that is eligible to prescribe prescriptions, such as nurse practitioners, optometrists, dentists, etc.

### MIE

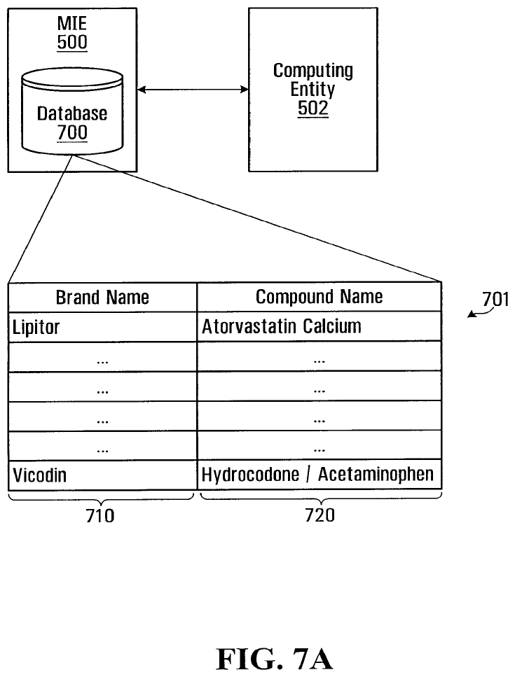

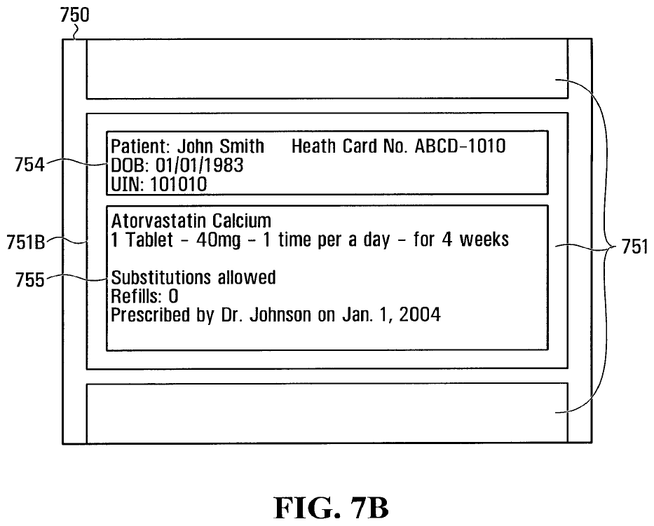

FIG. 7A illustrates a database 700 which stores data on the MIE 500. The database 700 may store a listing of brand name drugs and their corresponding chemical compound name or chemical formula and optionally the DIN associated with the drug. The table 701 of the database 700 may contain two columns the first column 710 lists brand name drugs and the second column 720 list the corresponding chemical compound. Alternatively the table may contain three columns where the third column contains the chemical formula corresponding the chemical compound and brand-name drug. For example, one of the rows in the table 701 may list Lipitor® in the first column 710 and in the second column 720 of the same row may list the corresponding chemical compound name Atorvastatin Calcium. A person skilled in the art would clearly understand that this table could contain thousands, tens of thousand, or even hundreds of thousand brand name drugs and their corresponding chemical compound, and the invention is not limited to the brand name drugs and corresponding chemical compounds listed in table 701. A person skilled in the art would also understand that there are numerous other ways to store this type of data in a database, and the current invention is not limited to such illustration. Optionally, the table 701 contains yet another column that includes DINs for the various drugs stored in the table 701.

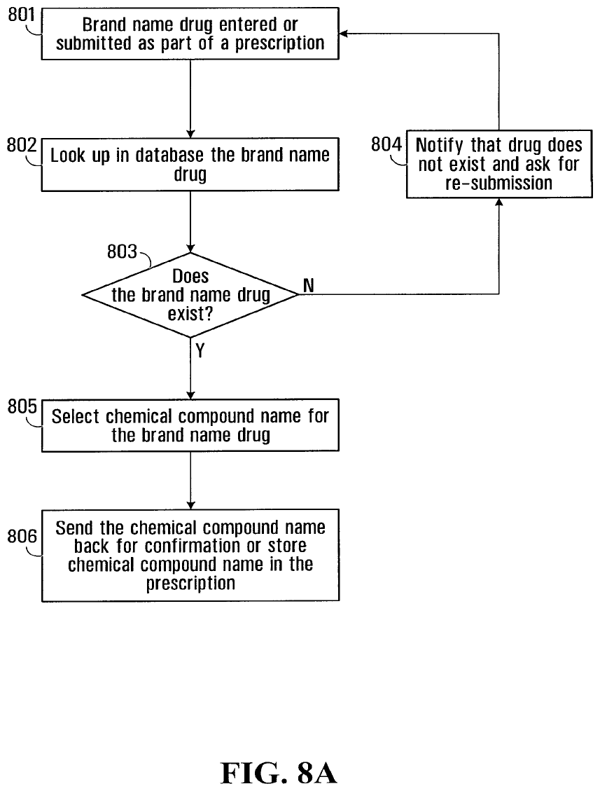

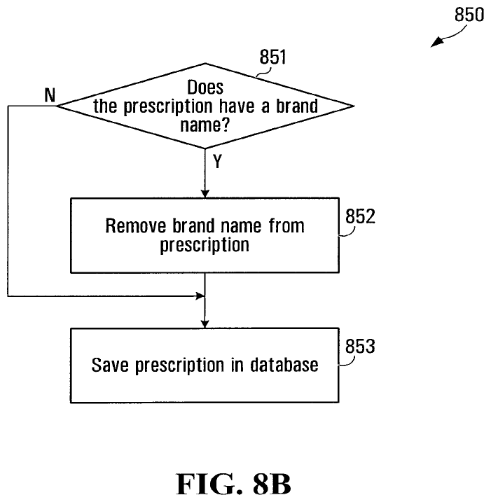

Referring now to FIG. 8A, when the physician at the computing entity 502 enters in a brand name drug to be looked up or to submit a prescription (step 801), the MIE obtains the brand name drug from the computing entity 502 and looks up in the database 700 the brand name drug (step 802). Then if no brand name drug by the name entered or submitted by the physician exists (step 803), then the MIE notifies the physician at the computing entity. This notification may be in the form of a pop-up box or other similar form of notification that the brand name drug does not exist and ask for a re-submission (step 804). For example, if the physician incorrectly types in or incorrectly spells a drug name the MIE would notify the physician computing entity 502 of the error and then ask for a re-submission with the correct name. If at step 803 the brand name drug does exist then at step 805 the chemical compound corresponding to the brand name drug is selected from the second column of the corresponding row of the database 701. In the event that a brand name drug is to be looked up (the first option, previously at step 801), the chemical compound is then sent back to the computing entity 502 for verification. In the event that a prescription with a brand name drug is submitted to the MIE (the second option, previously at step 801), the chemical compound is then stored in the electronic prescription for the electronic record associated with the individual that the prescription is for on the MIE.

The physician's computing entity 502 may submit a prescription in two forms (i) a prescription with the chemical compound of the brand name drug prescribed (ii) a prescription without the chemical compound of the brand name drug prescribed. As discussed above, if the prescription is submitted without a chemical compound the MIE would then lookup the chemical compound for the brand name drug prescribed and submit it back to the physician's computing entity 502 for verification by the physician. Upon verification the prescription would then contain a chemical compound.

The prescription which may contain the chemical compound name may be stored on the MIE in association with a record associated with the individual that the prescription is for. The prescription may include such information (which may be referred to as prescription information) as the patient identifier, the patient name, date of birth, age gender, weight, contact information, the provider or physician that prescribed the medication, etc., and the prescription information itself. The prescription information may include the medication name in the form of a brand name drug, the chemical compound name, the dosage, the duration, dispensing instructions, the number of refills (if any), whether substitutions are allowed, comments, the pharmacy that the prescription was sent to, the drug identification number (DIN), etc.

If substitutions are allowed the prescription stored on the MIE may remove the brand name drug from the prescriptions and only leave the chemical compound name. Alternatively, the MIE may not remove the brand name drug from the prescription.

For Example, FIG. 7B illustrates an example of a database table 750 stored in the database 700 containing a plurality of records 751. A specific record 751b, from the plurality of records, is shown having an identifier portion 754 corresponding to a specific patient (John Smith) and a prescription portion 755. The prescription portion 755 corresponds to a prescription that has the brand name drug associated with said prescription removed from it and contains a chemical compound name corresponding to said brand name drug. As is shown, the prescription portion 755 corresponds to the prescription prescribed by Dr. Johnson as illustrated in FIG. 6, with the brand name “Lipitor” removed. Referring now to FIG. 8B, the electronic prescription received at the MIE 500 from the computing entity 502 at the physician's office may be processed according to the method 850 to remove the brand name drug, if the electronic prescription is received at the MIE 500 having a brand name drug. At step 851, which may take place after step 806 of FIG. 8A, the MIE checks to see if the electronic prescription has a brand name drug associated with it. If the electronic prescription does not have a brand name drug associated with it, it is then stored in the database 700 on the MIE 500 in association with the patient that the prescription was prescribed for. If the electronic prescription does have a brand name drug associated with it, at step 852 the MIE removes the brand name drug from the prescription. At step 852, the MIE may check to see if substitutions are allowed and if substitutions are not allowed, the MIE in this case would not remove the brand name drug. After the brand name drug is removed, at step 853 the electronic prescription is saved in the database 700 on the MIE 500 in association with the patient that the prescription was prescribed for.

Although the above embodiment list the database to obtain chemical compound names on the MIE, in another embodiment it could be possible for the database to be located on a physician's computing entity or servers on a network that the physician's computing entity is apart of.

Furthermore, although the above embodiment is illustrated by use of a physician the above mentioned submission or entering of a prescription may take place at any professional's computing entity that is eligible to prescribe prescriptions, such as nurse practitioners, optometrists, dentists, etc.

### Server 501

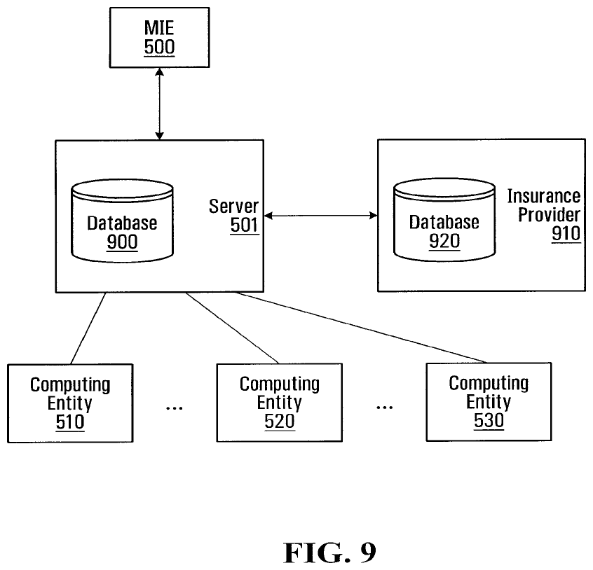

As noted above, the server 501 may be a server located at pharmacy, a server managed by a pharmacy chain, or a server managed by another service provider. As illustrated in FIG. 9, the server 501 may contain a database 900. The database 900 may store data which lists all of the brand name and generic name drugs available for a specific chemical compound name.

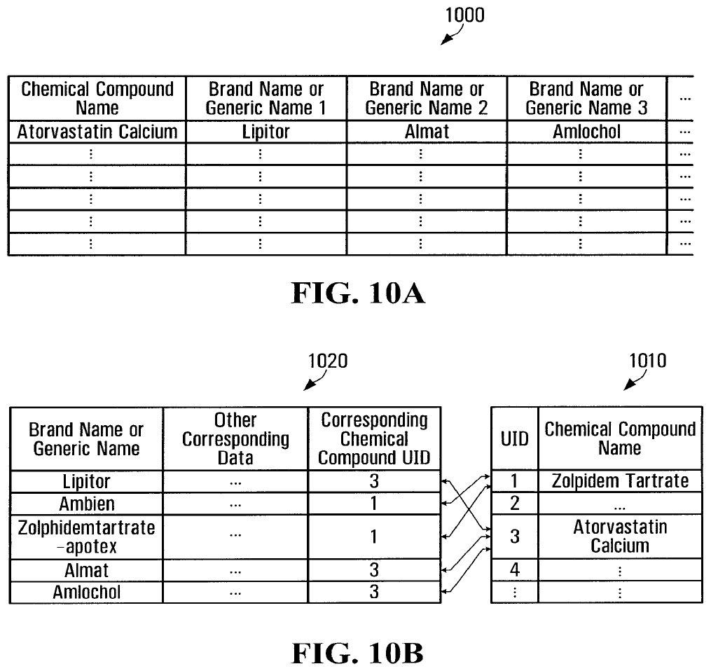

By way of example, FIG. 10A shows a table or data structure which lists chemical compounds in a column and then several other columns which list brand name or generic name drugs and corresponding data. That is, a single row of table or data structure 1000 would list a chemical compound in the first column and then all the cells in the row would contain brand name drugs or generic drug names and corresponding data. The corresponding data could include such information as the price of the drug, the stock at the pharmacy, which drugs are on sale, etc.

By way of another example FIG. 10B shows two database tables where table 1010 lists the chemical compounds along with a unique identifier (UID) and where table 1020 lists brand name or generic name drugs along with other corresponding data and the UID of the chemical compound. The other corresponding data field could include such information as the price of the drug, the stock at the pharmacy, which drugs are on sale, etc. Furthermore, although only illustrated as a single column in FIG. 10b, the other corresponding data column could be more than one column in the table. A person skilled in the art would understand that this two table structure with the UID would allow the database to search or query the tables to find all available brand name or generic name drugs for a chemical compound or to find the chemical compound associated with a brand name or generic name drug.

A person skilled in the art of databases would understand that there are numerous ways to implement the database that stores chemical compound and corresponding brand name or generic name drugs and the corresponding data, and the database would not be limited to the tables or data structures discussed above.

Although the above embodiments the database 900 is located on the server 501, in another embodiment it could be possible for the database 900 to be located on the MIE or on others servers on a network that the MIE is apart of. Furthermore, the server 501 may even be located on a pharmacy's computing entity.

It will be appreciated that the database 900 which stores data that lists all of the brand name and generic name drugs available for a specific chemical compound name can be accessed by a computing entity at a pharmacy to determine all brand name and generic name drugs associated with a chemical compound name when an electronic prescription is obtained from the MIE that only has a chemical compound name associated with it (i.e., when a prescription is stored in the MIE's database without having a brand name drug).

### Computing Entity at Pharmacy

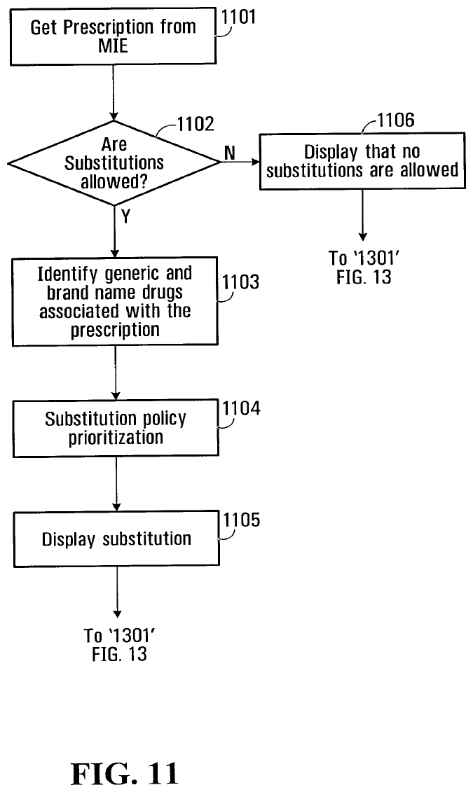

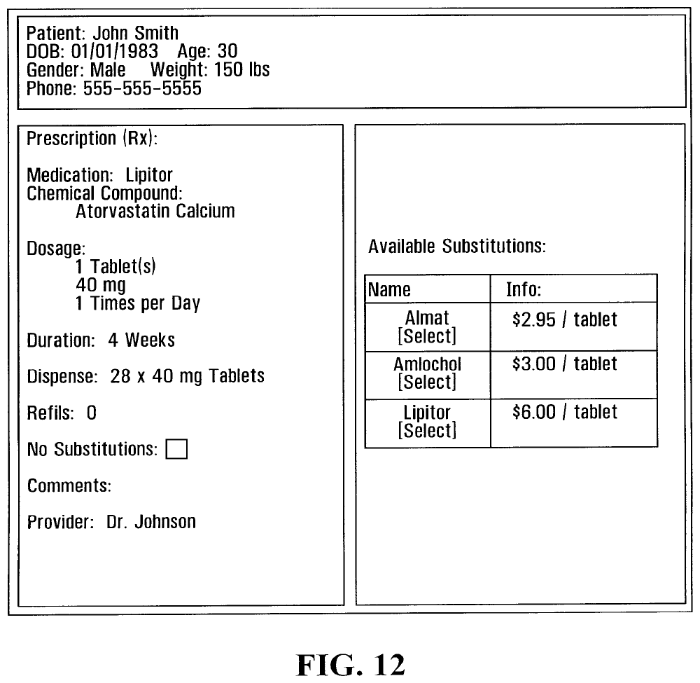

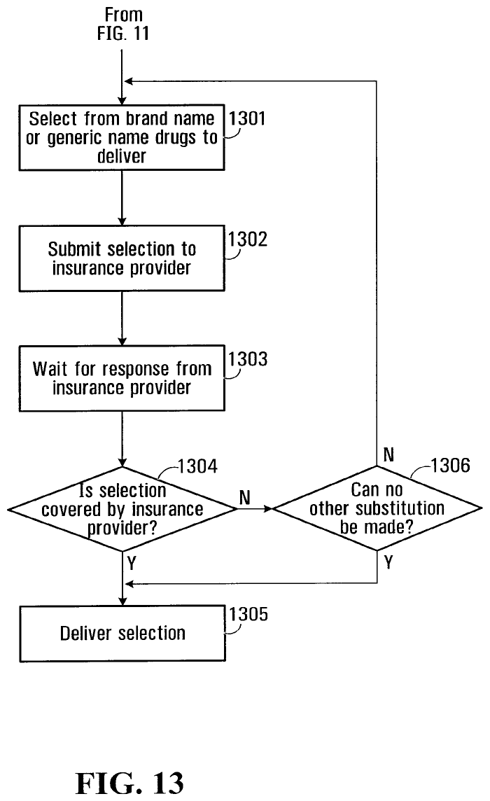

FIG. 11 is a flowchart of a process which is implemented by software executing on the computing entity at the pharmacy. The computing entity 510 which is located at a pharmacy gets a prescription from the MIE upon the request of a pharmacist (step 1101). As discussed in other embodiments of the invention the pharmacist at the computing entity would have to identify himself to the MIE and the MIE would have to validate his credentials, before requesting a prescription from the MIE. The pharmacist at the computing entity 510 would then check to determine if substitutions are allowed or not. If substitutions are not allowed this would be visible on the UI of computing entity 510, then the pharmacist selects the prescription as prescribed by the physician (FIG. 13, step 1301). If substitutions are allowed, as indicated in the prescription prepared by the physician and stored in the MIE, at step 1103 the computing entity would then get all of the generic and brand name equivalents for the prescribed drug. This may involve connecting or communicating with the server 501 which stores in a database 900 all brand name and generic drugs for a specific chemical compound. This may also involve querying the database for a request for certain information. For example, a request may be to return all brand name and generic name drugs for a specific chemical compound that would be available for substitution. By way of another example, a request may be to return all generic drugs available for a prescribed brand name drug that would be available for a substitution. Upon making this request for the names of the drugs that are available for substitution, the server 501 may prioritize the list of available substitutions (step 1104). Alternatively, the prioritization may occur on the computing entity 510. The prioritization of the available substitutions may be based on a number of factors such as: the stock of the drug by the pharmacy; the price; whether the drug is currently on sale or a rebate is offered; the patients past preference; etc. The list of available substitutions is then displayed on the UI on the computing entity 510 (step 1105).

For example, the substitution policy prioritization may include looking at a profile of the patient stored in a database located on the computing entity 510, the server 501 or the MIE 500 to determine which brand or generic drug the patient has previously taken. The patients profile may include information such as which drugs the patients insurance provider will accept. For example, if the patient's insurance provider only covers the cost of generics then this may be stored in the patients profile and assessed when determining the prioritization of the list of brand name and generic drugs to be displayed on the UI. By way of another example, if the pharmacy is currently out of stock of a certain brand name or generic drug name this certain brand name or generic drug name may not be listed on the UI, or may be listed at the bottom or this list, or may be listed with an indicator that this brand name or generic drug name is currently out of stock. Furthermore, the pharmacy or chain of pharmacies may get a discount or a rebate on certain drugs, or may want to promote a specific brand name or generic drug name, and these factors would be taken into account when prioritizing the list of drug names to be displayed on the UI. For example, FIG. 12 shows the UI which shows the patients prescription as obtained from the MIE and the available substitutions based on a substitution policy prioritization.

At step 1301 the pharmacist selects the brand name or generic drug name to be prescribed from the list of available substitutions and then if the patient has insurance, submits (step 1302) the prescription information which may include the substituted drug to the insurance company. The information submitted to the insurance company may also include the patient's name, date of birth, etc. and/or a policy id or plan id number. The submission to the insurance provider may be made by way of a connection to an insurance provider's server 910 which may have a database 920. Upon submission to the insurance provider, the computing entity 510 may then wait for a response (step 1303). The insurance provider's server 910 may then determine whether the patient's policy covers the prescribed drug. If the patient's policy covers the prescribed drug the insurance provider's server 910 may notify the computing entity 510 that such a prescription is covered by the policy (step 1304). Reimbursement from the insurance provider to the pharmacy may then also occur at this time. The pharmacist then may deliver the drug (step 1305).

If at step 1304 the insurance provider does not cover the prescribed prescription or only covers part of the prescription the UI of the computing entity receives a notification that the prescription is not covered or only partly covered. The pharmacist may then determine what to do such as selecting another brand name or generic named drug (back to step 1301) and resubmit (step 1302). Alternatively, the patient may want a specific brand or the physician may have indicated that no substitutions are allowed, in this case the pharmacist can then delver the drug but receive payment from the patient for the amount not covered by the insurer or the whole amount if the patient's policy does not cover the prescribed drug. Reimbursement from the insurance provider to the pharmacy may then also occur at this time for the part, if any, that is covered by the insurance company.

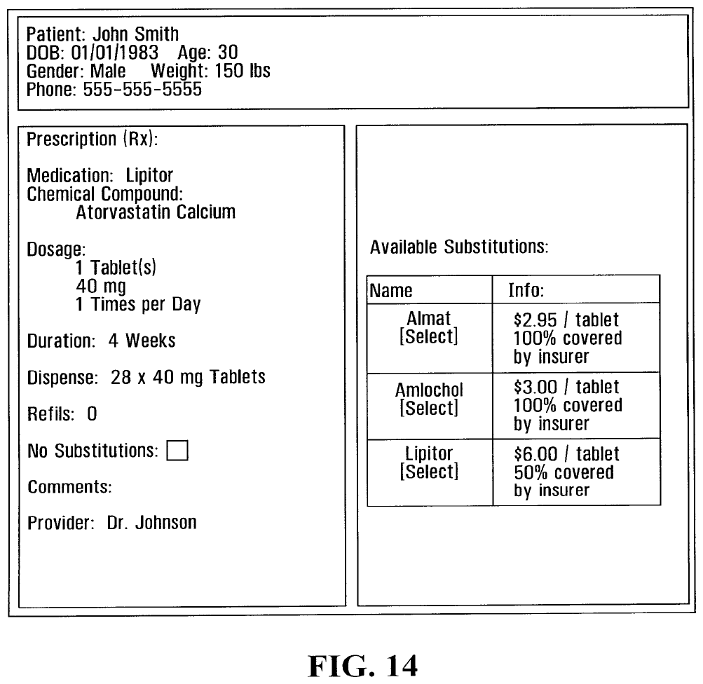

Alternatively, this whole process of submitting a drug and re-submitting may be automated. That is, the computing entity 510 or server 501 may retrieve from the insurance company's server 910 the patients policy information that indicates what the insurance company will and will not cover. The computing entity 510 or the sever 501 may then prioritizing the list of brand name or generic name drugs to display as available substitutions and prioritize or list only brand name or generic name drugs that will be accepted by the insurance company for the patient. For example, if the insurance company will not cover the cost of the brand name drug this may be indicted in the list of available substitutions. For example, FIG. 14 shows a prioritized substitution list at the UI of computing entity 510 where the generic's Almat and Amlochol are fully cover by the patient's insurance plan, while the brand name drug is only 50% covered.

Another alterative to the automated process to determine whether the available substitutions are covered by the patient's insurance policy is for the computing entity 510 or server 510 to communicate the list of prioritized available prescriptions to the insurance company server. The insurance company server then may determine which brand name or generic drugs are covered by the patient's policy and submits this information back to computing entity 510 or server 510. The computing entity 510 or server 510 may then re-prioritize the results before being displayed on the UI. The pharmacist may then select from the UI which brand name or generic drugs to substitute for.

A person skilled in the art would understand that the steps of the flow chart of FIG. 11 and FIG. 13 may be in a different order.

Although the above embodiment is illustrated by use of a pharmacist the above mentioned embodiment may utilized by other professionals such a pharmacist technician, druggist, etc.

Furthermore, parts of the process discussed above may occur at either the server 501 or any of a number of computing entities.

### EXAMPLE 1

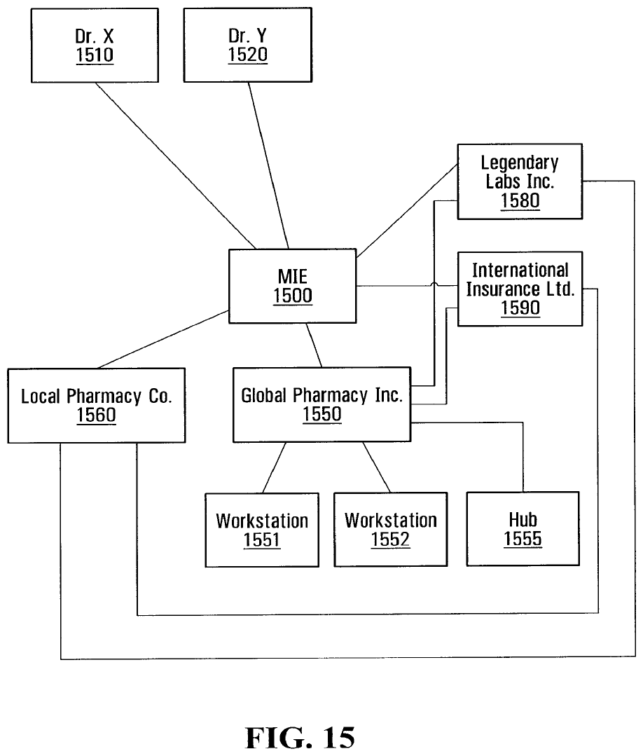

A specific, simplified, non-limiting example will now be provided using the connectivity of the computing entities, servers, and MIE as illustrated in FIG. 15.

Although in FIG. 15 the MIE, the computing entities, and servers are identified by different reference characters in this example than previously identified, the MIE, the computing entities, and servers are only reference by these different reference characters to identify them for this example and may be the same and/or configured in the same way as the MIE, the computing entities, and servers discussed elsewhere in this document.

In this example, there are four patients: Adam Smith, Brittany Johnson, Carl Adams and Doug Thomas. Each of these patient has previously given consent to have their medical record on MIE. Furthermore, in this example, the MIE is managed by an organization identified as the Provincial Health Record. Between these three patients, Adam Smith, Brittany Johnson visit Dr. X and Carl Adams and Doug Thomas visit Dr. Y to get medical prescriptions which are registered with the MIE. After receiving their prescriptions each of these patients visit a pharmacy either Global Pharmacy Inc. or Local Pharmacy Co. to receive their medication where the pharmacies retrieve the prescriptions from the MIE. Furthermore, in this example, each of these patients is insured by International Insurance Ltd. Moreover, in this example, if any of these patients requires a laboratory test to be done, their tests are conducted by Legendary Labs Inc.

Referring to FIG. 15, Dr. X's computing entity 1510 and Dr. Y's computing entity 1520 are connected to the MIE 1500. Although not illustrated in this example, the connection may be through a server located at either office of Dr. X or Dr. Y. Dr. X has software running within his local network that has access to a database of brand name drugs and their chemical compound name equivalents. On the other hand, Dr. Y does not have access to a database that maps brand name drugs to their chemical compound name equivalents locally but is able to get this information from the MIE 1500.

Global Pharmacy Inc.'s server 1550 is connected to the MIE 1500. Furthermore, the server 1550 is connected to a USB key hub 1555 and two computing entities in the form of workstations 1551 and 1552. In this example, Global Pharmacy Inc. has two pharmacists with surnames Anderson and Brown. Anderson and Brown have each been provided with a USB key (i.e., a hardware token) from the Provincial Health Record. Anderson's USB key is associated with him and Brown's USB key is associated with her. At the beginning of each shift, Anderson and Brown insert their USB keys into the USB key hub 1555.

Local Pharmacy Co. has a computing entity in the form of a workstation 1560 connected to the MIE. In this example, Local Pharmacy Co. has one pharmacist with the surname Clark. Clark has been provided with a USB key from the Provincial Health Record, where the USB key is associated with her. Unlike Global Pharmacy Inc., Clark inserts her USB key directly into the USB port on the workstation 1560.

In this example, every patient record has a unique identification number (UIN) for which the patient record is stored with in the MIE. The record also has other information including the patient's name, date of birth, sex, and health card number. Each record also includes prescription information, lab test information, medical conditions and allergies, and insurance information.

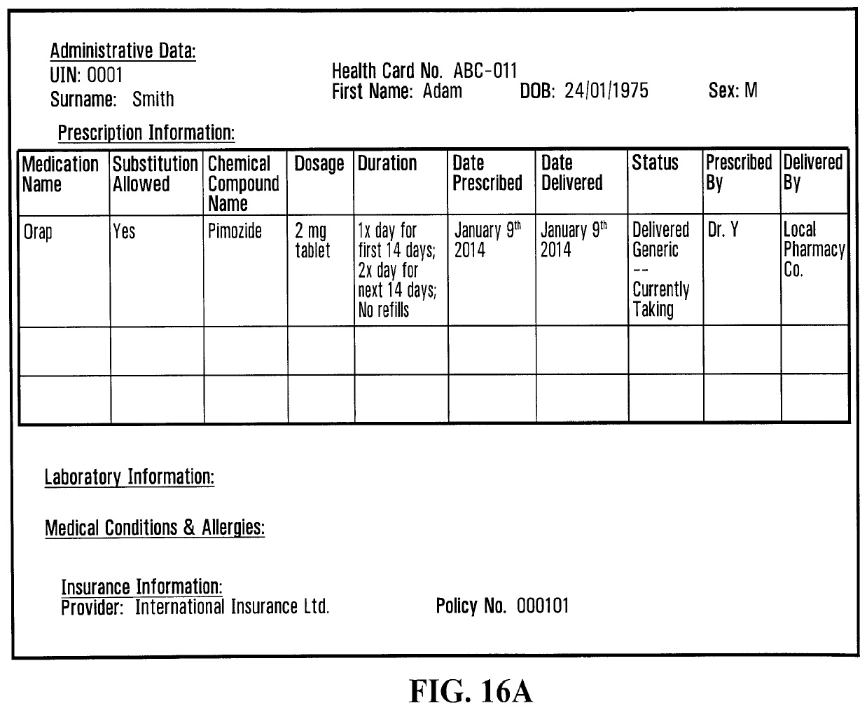

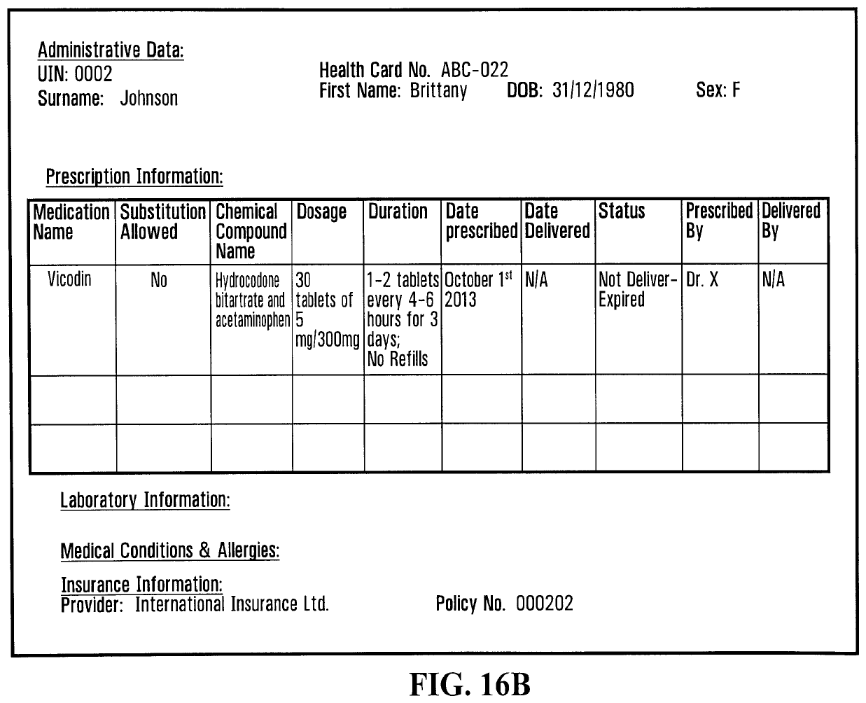

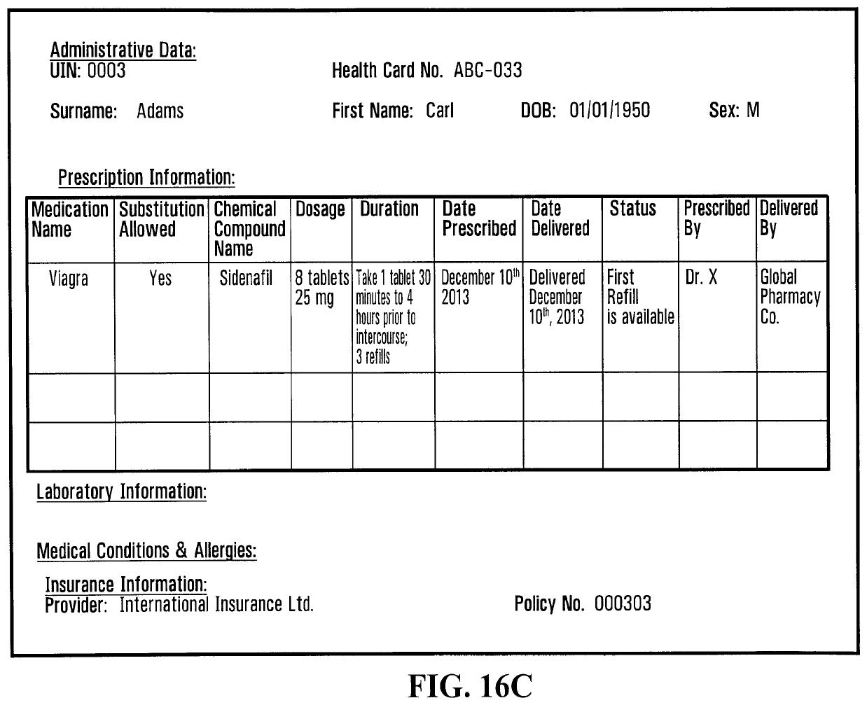

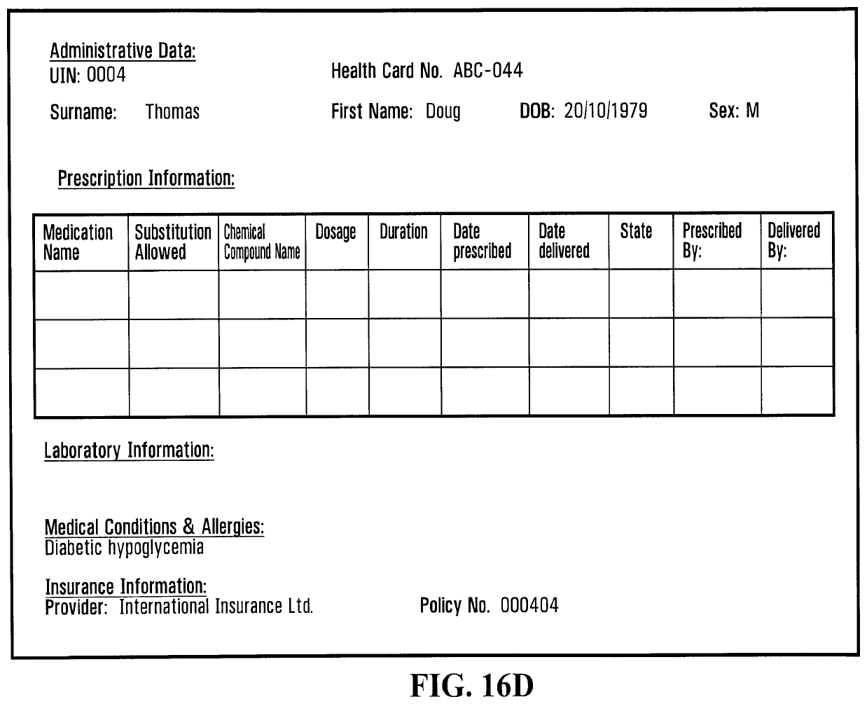

Simplified versions of the medical records of Adam Smith, Brittany Johnson, Carl Adams, and Doug Thomas are shown in FIGS. 16A-16D. More specifically, Adam Smith's medical record is shown in FIG. 16A, Brittany Johnson medical record is shown in FIG. 16B, Carl Adams medical record is shown in FIG. 16C, and Doug Thomas medical record is shown in FIG. 16D.

For this example, it will be assumed that the current day is Jan. 10, 2014.

Referring now to FIG. 16A, Adam Smith's medical record indicates that Dr. Y on Jan. 9, 2014 prescribed a prescription for Pimozide which was delivered the same day.

Referring now to FIG. 16B, Brittan Johnson's medical record indicates that received a prescription for Vicodin® which was never delivered.

Referring now to FIG. 16C, Carl Adams's medical record indicates that received a prescription for Sildenafil and that a refill is available.

Referring now to FIG. 16D, Doug Thomas's medical record indicates that he has diabetic hypoglycemia.

In FIGS. 16A-16D the prescription information is shown in the record itself. However, this is for this simplified example and the prescription information may actually be stored in a separate server that is either a part or not part of the MIE and its network.

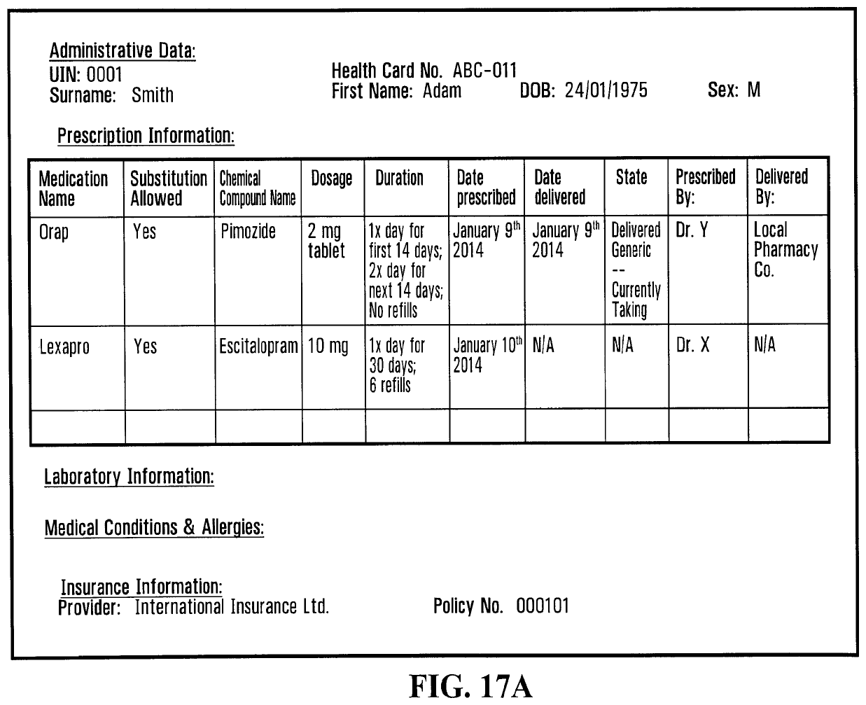

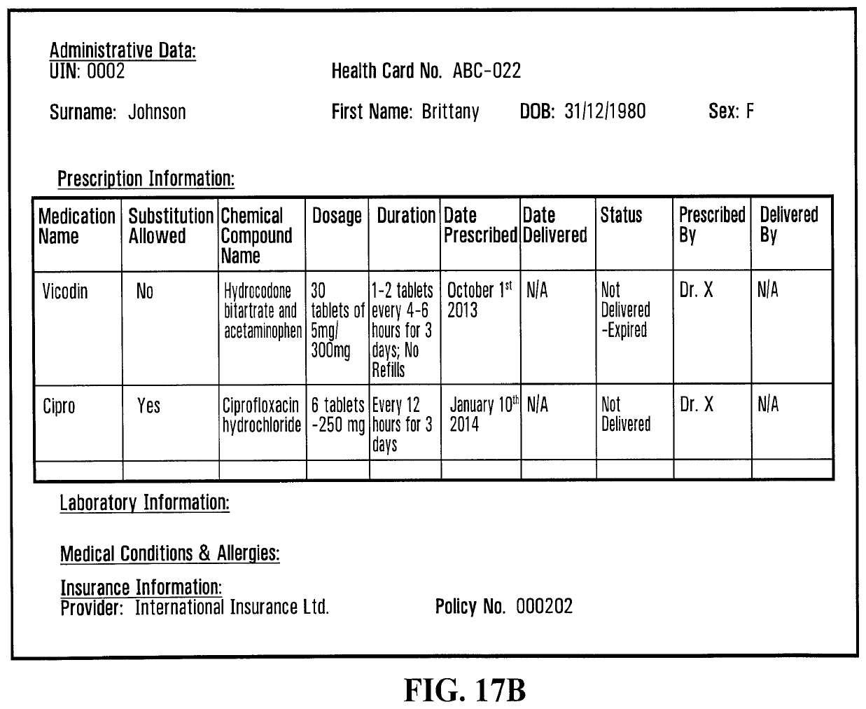

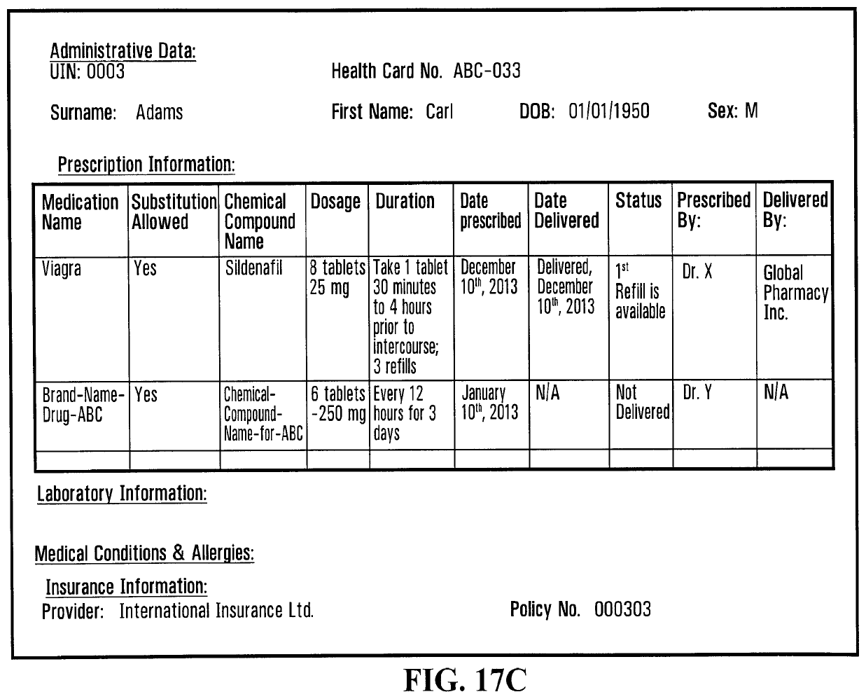

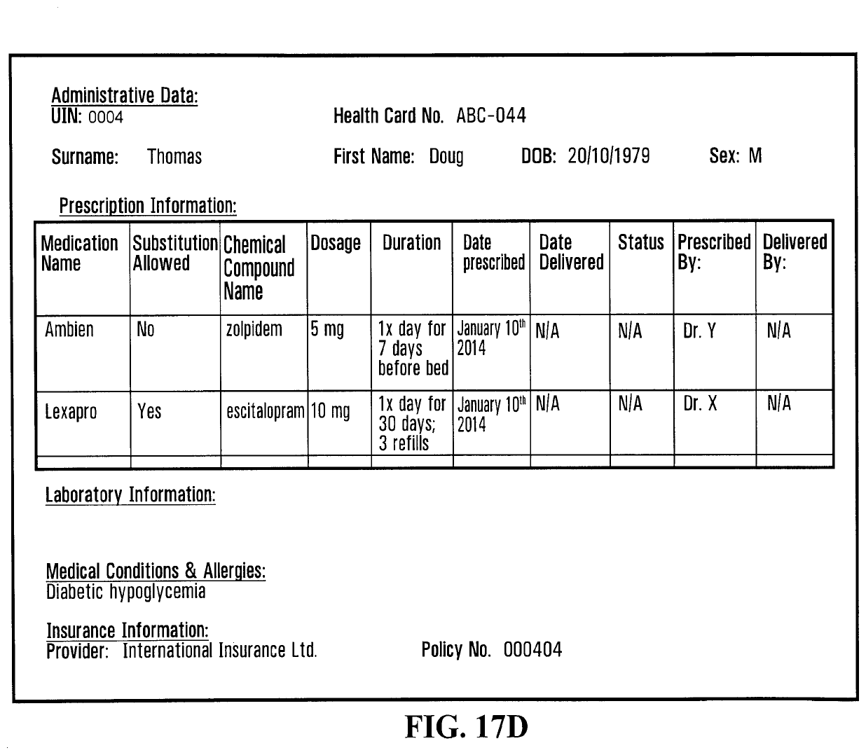

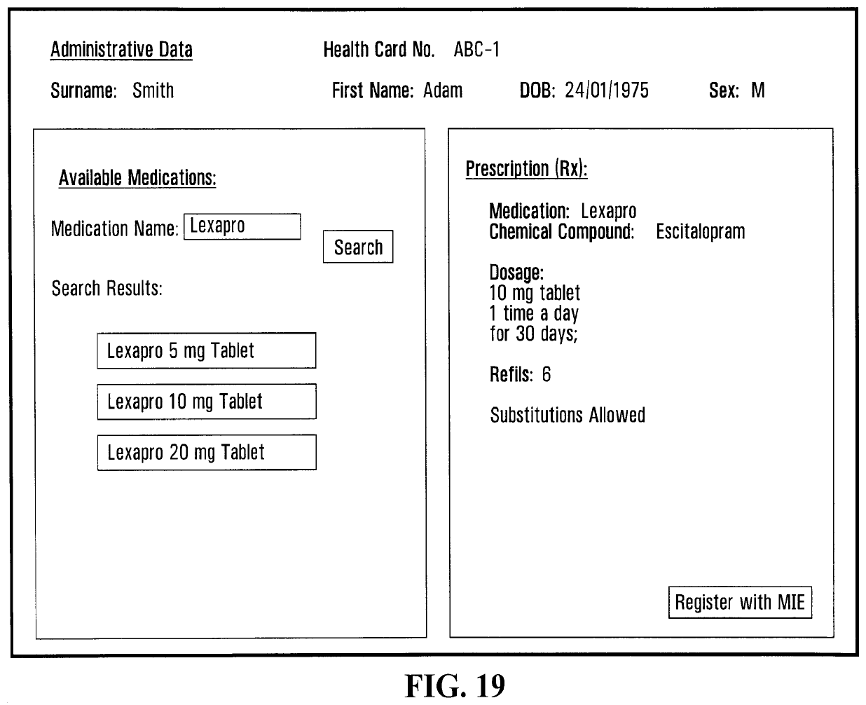

Now, on January 10^{th}, Adam Smith visits Dr. X and is prescribed a prescription for Lexapro® to help Adam with depression. As Dr. X's computer software has access to a local database for mapping a chemical compound name from a brand name drug, the chemical compound names for Lexapro® is Escitalopram obtained locally. FIG. 19 illustrates a screen shot of the software running on Dr. X's computing entity which illustrates the auto-completion of the chemical compound name as Dr. X selects Lexapro®. That is, as Dr. X selects the Lexapro® tablet size that he would like to prescribe from the list of available medications after searching for “Lexapro”, the Prescription (Rx) is loaded with both Lexapro® and Escitalopram. Then Dr. X is able to include the chemical compound name in the prescription that he sends/registers with the MIE. FIG. 17A illustrates Adam Smith's medical record after the new prescription has been registered with the MIE. In this example, Dr. X did not look at the MIE to see if Adam Smith was taking any other prescriptions (Although, Dr. X could have consulted the patient's record on the MIE).

Brittany Johnson visits Dr. X, also on January 10^{th}. Dr. X prescribes Brittany Cipro® tablets, as Brittany has a urinary tract infection. As Dr. X's computer software has access to a local database for chemical compound names for Cipro® which is ciprofloxacin hydrochloride, Dr. X is able to include the chemical compound name with the prescription that he sends/registers with the MIE (similar to the case discussed above). FIG. 17B illustrates Brittany Johnson's medical record after the new prescription has been registered with the MIE.

Carl Adams visits Dr. Y on January 10^{th} and receives a prescription for Brand-Name-Drug-ABC. Dr. Y's computer software does not have local access to a database that maps a brand name drug to the chemical compound name. As such, after Dr. Y registers/sends the MIE the prescription with the brand name drug, Dr. Y is notified to confirm the chemical compound name for the submitted drug (Alternatively, Dr. Y could access the database of the MIE and retrieve the chemical compound name for the brand name drug and then submit/register the prescription with the MIE). In other words, Dr. Y communicates with a database on the MIE to obtain and confirm the chemical compound name for the brand name drug. FIG. 17C illustrates Carl Adams's medical record after the new prescription has been registered with the MIE.

Doug Thomas visit Dr. Y on January 10^{th} and receives a prescription for Ambien® and Lexipro®. Similar to the case above, Dr. Y registers the prescriptions with the MIE and obtains/confirms the chemical compound names for the brand name drugs from the MIE. FIG. 17D illustrates Carl Adams's medical record after the new prescription has been registered with the MIE.

In this example, each of the four patients Adam Smith, Brittany Johnson, Carl Adams and Doug Thomas visit one of the pharmacies discussed above on the same day that they visited the doctor. These patients' interactions with the pharmacies and the pharmacies interaction with the MIE will now be discussed in more detail.

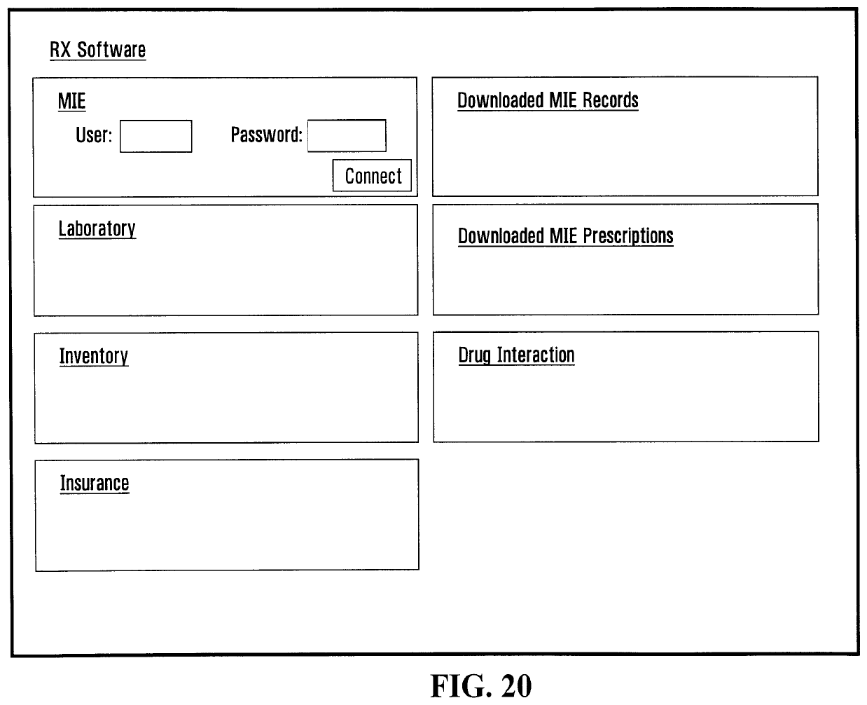

Adam Smith goes to Global Pharmacy Inc. which has one pharmacists Anderson currently working. At the beginning of his shift Anderson inserted his USB key into a port on the USB hub. Anderson then goes to workstation 1551 and login into the workstation. For the sake of this example, the workstation is running a single piece of software referred to as RX Software, however, this software may actually be multiple pieces of software (such as, MIE access software, local clientele management software, drug interaction software, laboratory submission software, inventory management software, etc.). After login into the workstation, Pharmacist Anderson then selects in the RX Software that he would like to interact with the MIE. This is done by Anderson selecting his name and is then prompted to enter in his password to access the MIE. FIG. 20 illustrates a screen shot of the RX Software before Anderson has selected his name and typed in his password. Then Anderson can click “connect”. A handshake occurs between the MIE and the workstation (including the USB key) to verify or validate that Pharmacist Anderson is able to access the MIE.

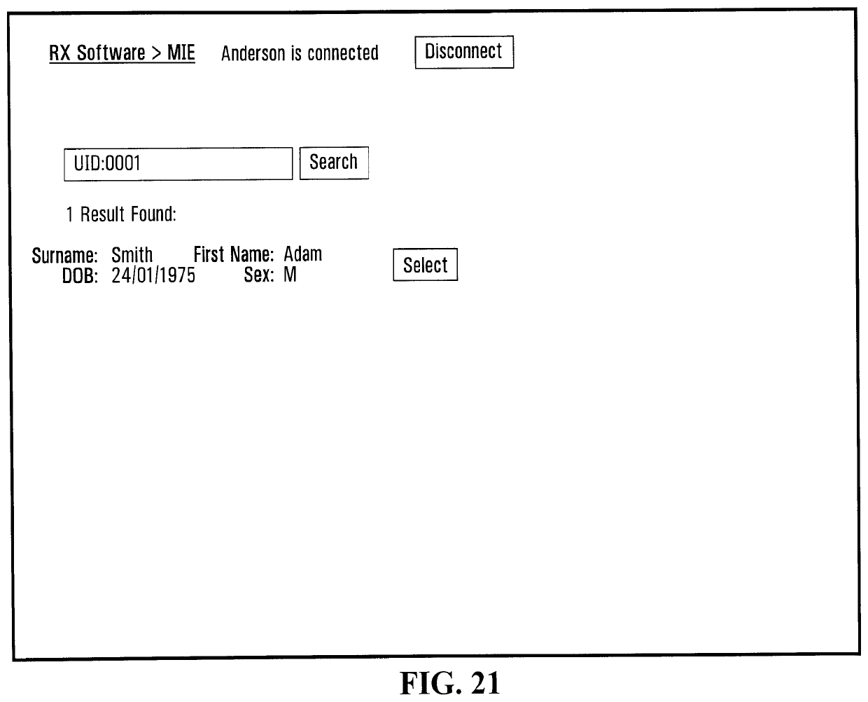

Adam Smith then tells Pharmacist Anderson that he has a prescription and provides Pharmacist Anderson with his MIE's UIN (“0001”). Pharmacist Anderson then clicks on the “MIE” tab of FIG. 20, which then brings him to the screen illustrated in FIG. 21. In FIG. 21, Pharmacist Anderson using the UIN provided (“0001”) is able to located Adam Smith's medical record from the MIE and to request and downloads his undelivered prescriptions and prescription history to the pharmacy's server and computing entities.

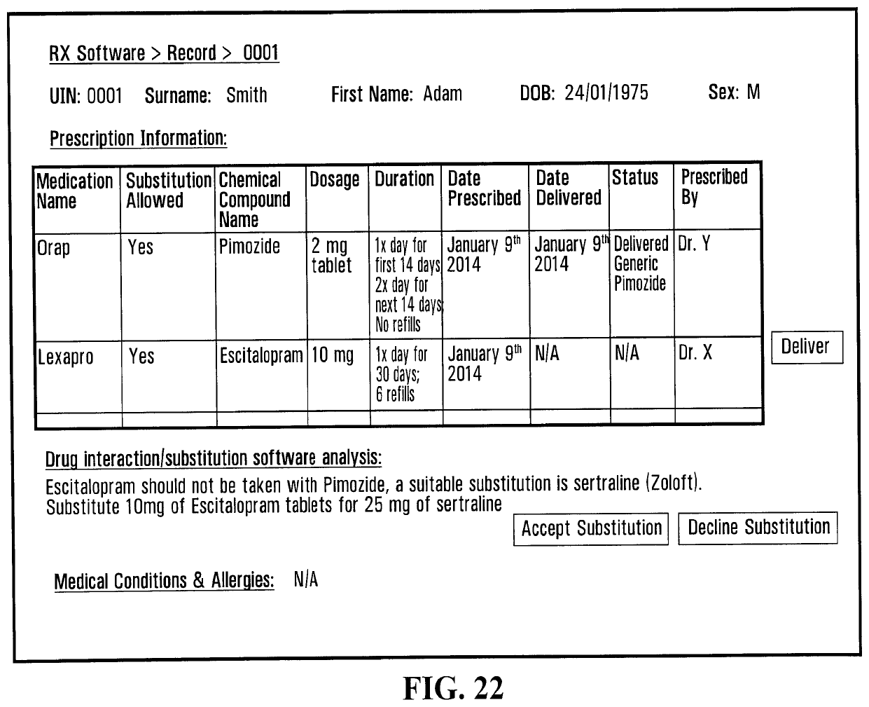

To view Adam Smith's record, Pharmacist Anderson clicks on the “Downloaded MIE Records” tab of FIG. 20. Then Pharmacist Anderson selects Adam Smith's prescription record. FIG. 22 shows the prescriptions history and available prescriptions to be delivered. Pharmacist Anderson runs the drug interaction software which notes that Escitalopram should not be taken with pimozide and the Escitalopram should be substituted to sertraline (Zoloft®). Pharmacist Anderson seeing the substitution can then accept or decline the substitution. In this case, pharmacist Anderson accepts the substitution. It is appreciated that the drug interaction software determines that based on the drugs that the patient is actively taken (i.e., drugs that have been both prescribed and delivered). Although in this example the drug interaction is determined by the pharmacist, in other cases, the drug interaction could be determined by the doctor prior to prescribing the prescription. In other words, when the doctor prescribes a prescription the doctor may be able to access the patients record stored on the MIE and determined which drugs that patient is actively taking.

As a result of this substitution, the previous prescription is cancelled and a notification of the cancellation of the prescription is sent to the MIE. A new prescription is then created for sertraline and the MIE is notified of the update. The modifications made to the prescription are stored in the database of the MIE. In other cases, the pharmacist may not be able to make the substitution without authorization from a doctor. In these cases, the pharmacist may send information back to the MIE that records the drug interaction (or other reason that the drug should not be subscribed) which could then be reviewed by the prescribing doctor (or a different doctor) which could then change the prescription to include a more suitable drug. In some cases, the information sent back to the MIE could include a drug that the pharmacist recommends based on the drug interaction (or other reason that the drug should not be subscribed) which can then be accepted or declined by the doctor.

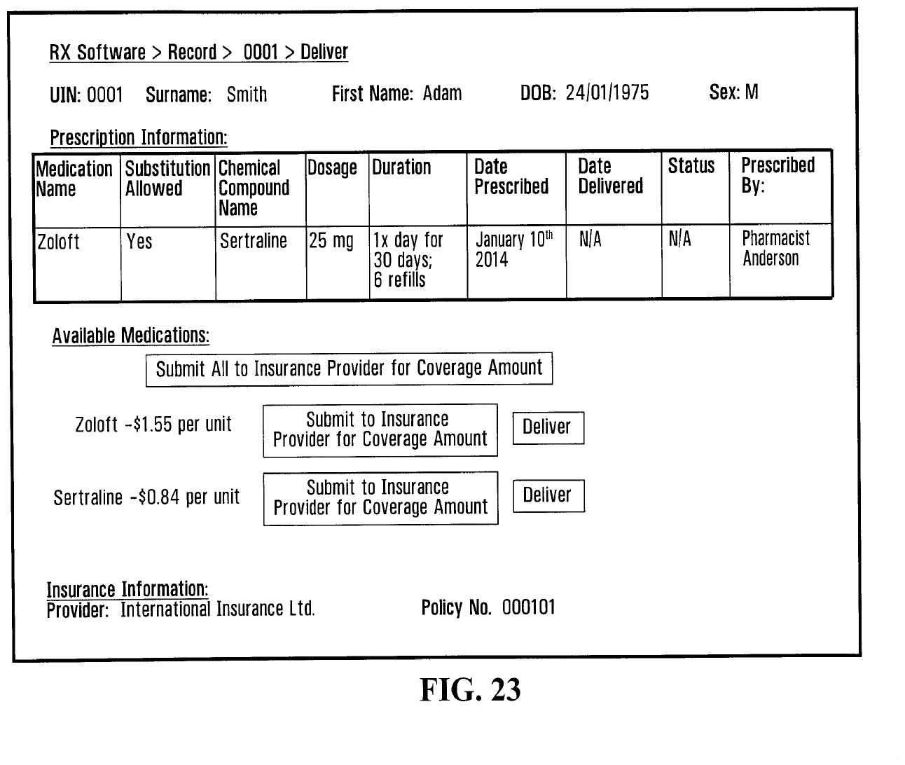

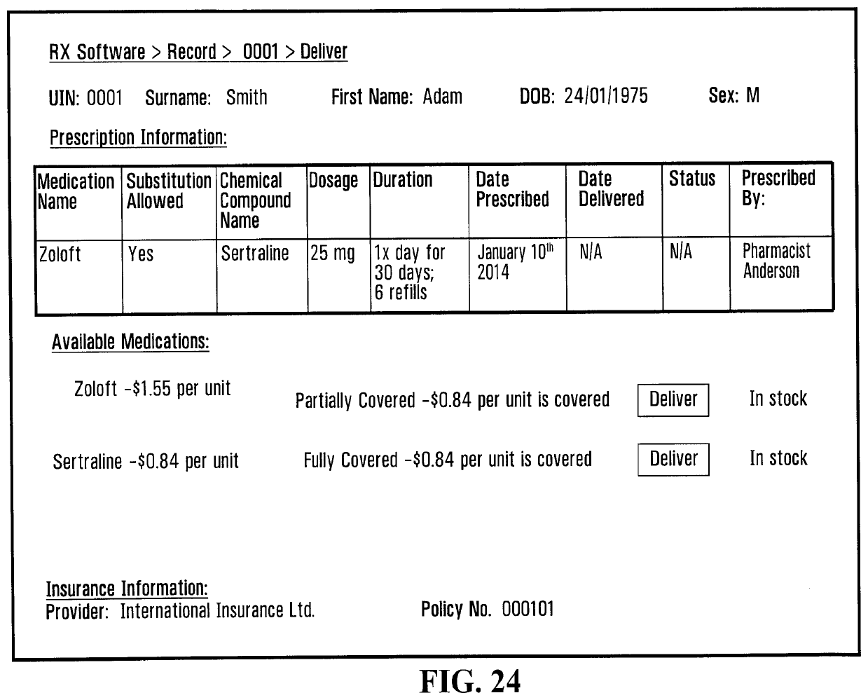

In this case, Adam Smith has already provided his insurance information (i.e., that he is insured by International Insurance Ltd. and his policy number) and this information is stored in his record. Pharmacist Anderson can then determine whether to deliver the brand name version of the drug or which generic version of the drug based on factors such as price and insurance coverage. FIG. 23 shows the screen of the Rx Software where the pharmacist can select to deliver the generic or the brand name drug and submit the available drugs to the insurance provider to see what amount is covered. As illustrated in the figure, the screen shows the price for Zoloft® and the price for Sertraline. Here, Pharmacist Anderson selects “Submit All to Insurance Provider for Coverage Amount”. FIG. 24 shows the resulting prices per a unit for Zoloft® and Sertraline. In this example, only the price of generic is covered, which is $0.84 per a unit. Pharmacist Anderson then selects “Deliver” beside the generic Sertraline. Next, Pharmacist Anderson prepares the prescription and delivers it to Adam Smith.

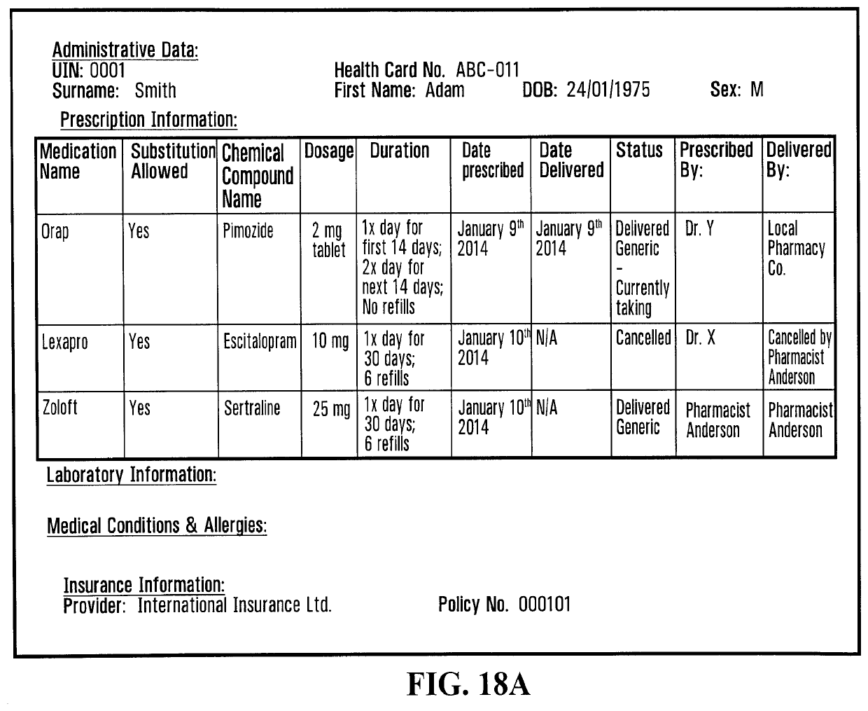

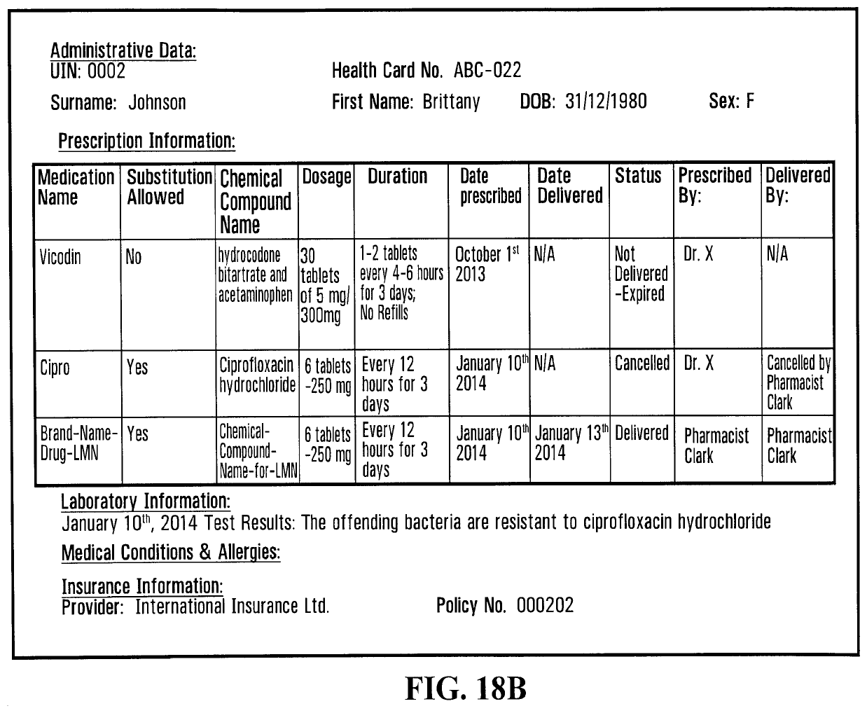

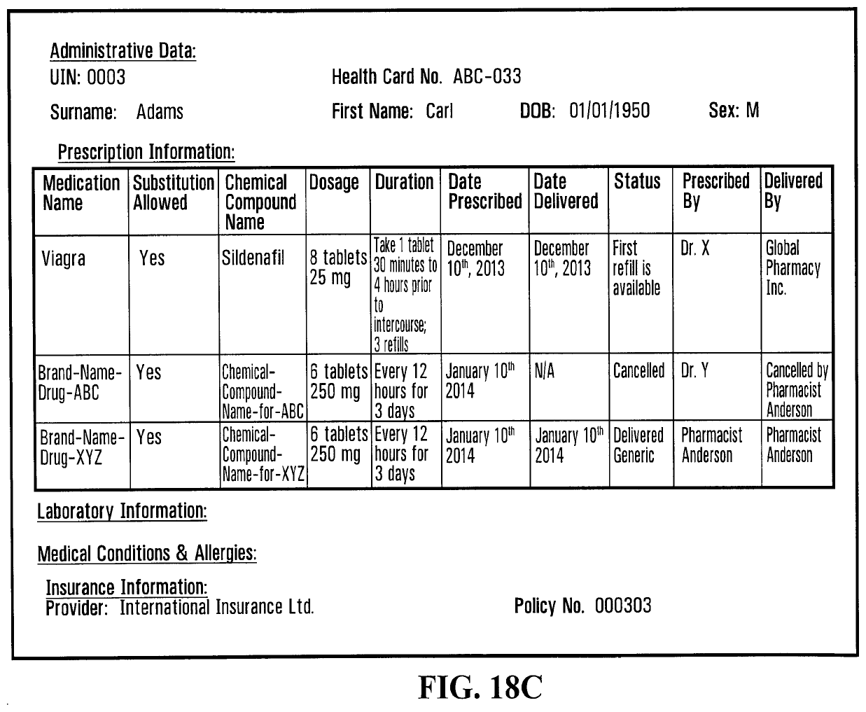

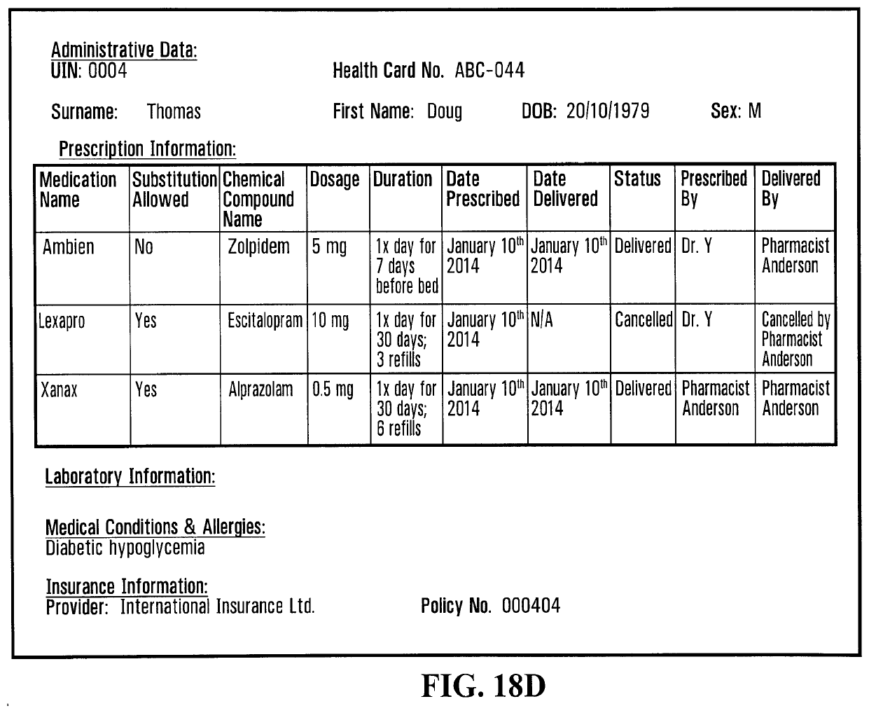

The record of the delivery of the prescription is sent to the insurance company so that the pharmacy can receive payment. Furthermore, the MIE record is also updated to reflect the changes of the delivered prescription. In other words, prescription fulfillment information indicating that the prescription has been fulfilled is sent back to the MIE to be stored in the patient's record. FIG. 18A illustrates the updated record of Adam Smith at the MIE after the prescription has been delivered. The record still retains the previous prescription for Lexapro® but it is changed to indicates that it was cancelled.

Turning now to Brittany Johnson, she goes to Global Pharmacy Inc. which now has two pharmacists working. Just prior to Brittany Johnson's arrival, Pharmacist Brown started her shift. At the beginning of her shift Brown inserted her USB key into a port on the USB hub. Now the USB hub has two USB keys currently inserted, one for Brown and one for Anderson. Brown then goes to workstation 1552 and logs into the workstation. The process of Brown login into the workstation is similar to that of Anderson. After login into the workstation, Pharmacist Brown then selects in the RX Software that she would like to interact with the MIE. Similarly, Brown connects to the MIE by selecting her username and entering in her password and a handshake process occurs. Once validated, Brown is able to the process prescriptions for clients of the pharmacy.

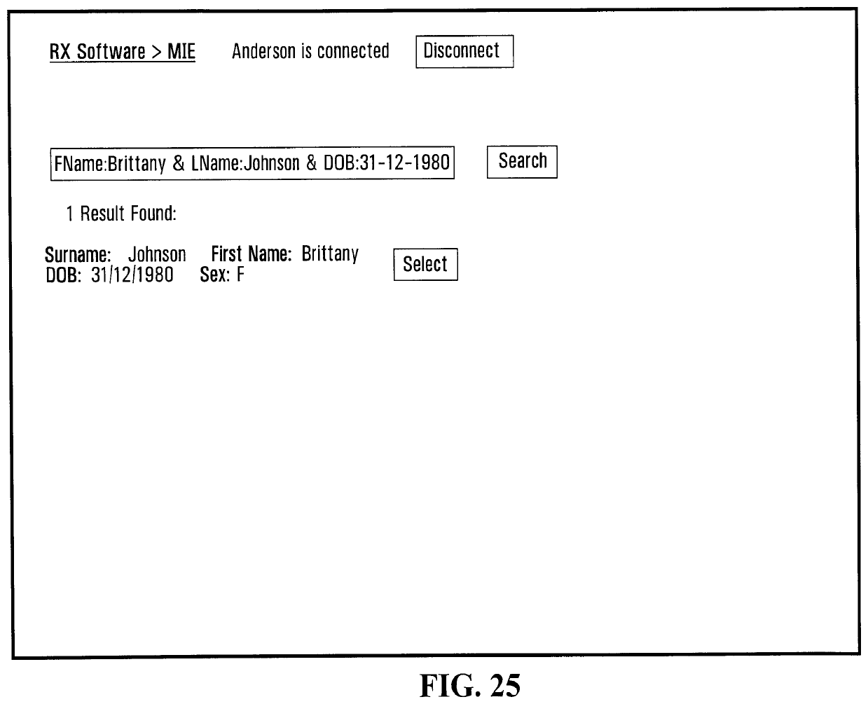

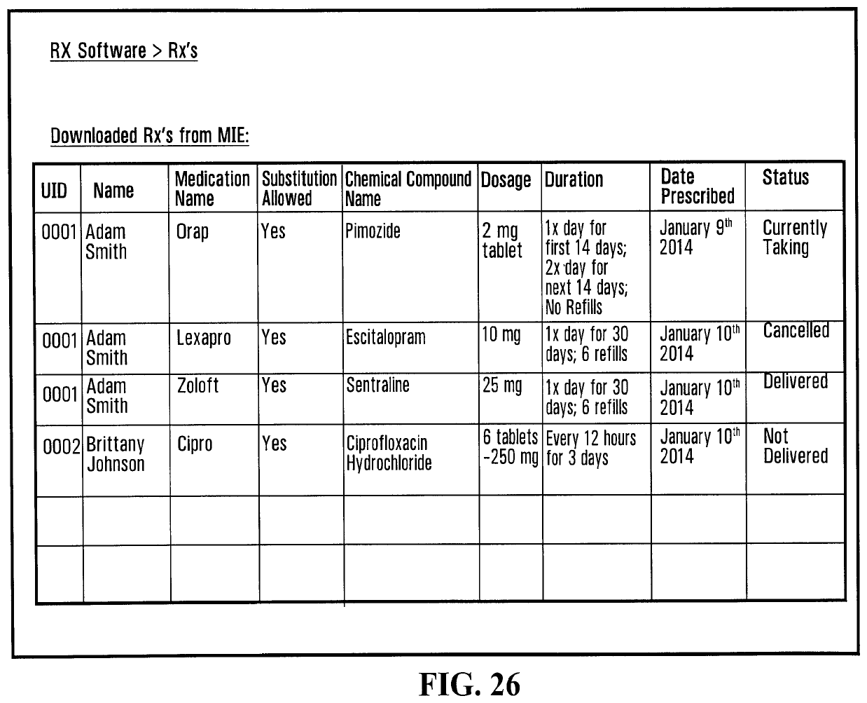

Brittany Johnson then tells Pharmacist Brown that she has a prescription and provides Pharmacist Brown with her name and date of birth (31 Dec. 1980), as she cannot remember her MIE UID. Pharmacist Brown then clicks on the “MIE” tab (of FIG. 20), which then brings her to the screen illustrated in FIG. 25. In FIG. 25, Pharmacist Brown using the date of birth provided and name is able to located Brittany Johnson's medical record from the MIE and retrieves her undelivered prescriptions and prescription history to the pharmacy's server and computing entities. Then, Pharmacist Brown clicks on the “Downloaded MIE Prescriptions” tab of FIG. 20. FIG. 26 illustrates all of the currently downloaded prescription to the Global Pharmacy Inc. network. More specifically, FIG. 26 shows all of the prescription for Adam Smith (currently taking, cancelled, and prescribe/delivered) and prescription for Brittany Johnson. As shown in FIG. 26, only prescriptions that are available to the patent, prescription that the patient is currently taking, or prescriptions that were prescribed and then cancelled locally by the pharmacy are shown in the list of downloaded prescriptions.

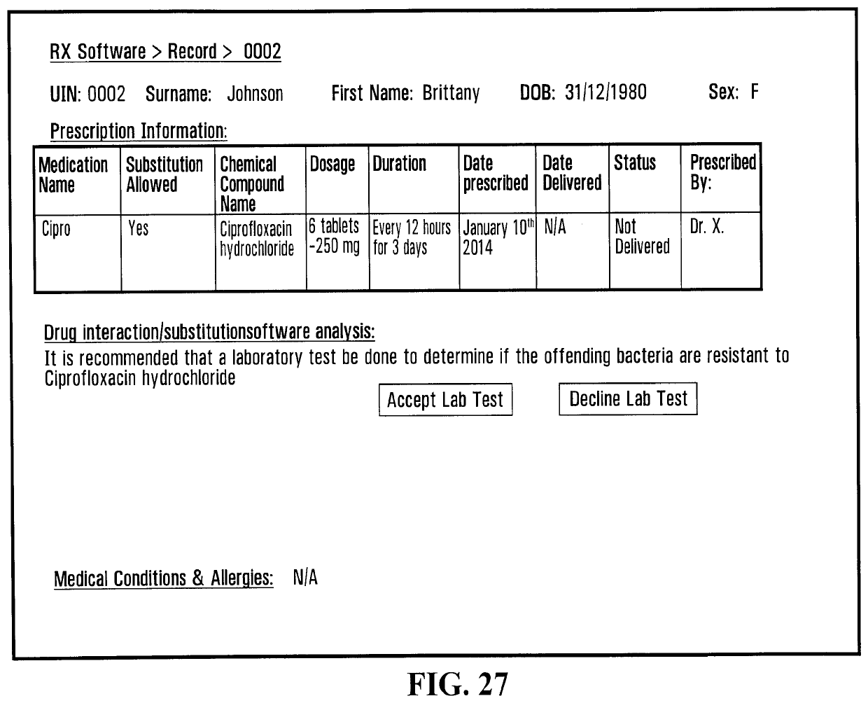

Pharmacist Brown selects the prescription for Brittany Johnson from the list of prescription in FIG. 26. Pharmacist Brown is then taken to the screen of FIG. 27 which shows the prescription to be delivered and that the drug interaction software recommends that a laboratory test be done to determine if the offending bacteria are resistant to Ciprofloxacin hydrochloride. In this case, Pharmacist Brown selects “Accept Lab Test” and instructs Brittany Johnson to go to a local laboratory to get a test done to determine if the offending bacteria are resistant to Ciprofloxacin hydrochloride and tells her to come back after the lab results are done. Brittany than leaves the pharmacy with the intention of visiting a laboratory later in the day.

Shortly after Brittany Johnson leaves the pharmacy, Carl Adams goes to Global Pharmacy Inc. and Pharmacist Anderson helps him. Similar to the customers before, Carl Adams asks for his prescription and provides his UIN. Pharmacist Anderson notices that a first refill of Carl Adams's prescription for Sildenafil is available and asks Carl Adams if he would like his prescription refilled. Carl Adams indicates that he would like this prescription refilled in addition to the prescription that was prescribed today by Dr. Y. Pharmacist Anderson then before starting to prepare Carl Adams's prescriptions checks the insurance coverage of both prescriptions. He first checks whether the generic Sildenafil is covered by Carl Adams's insurer, which it is. Then he checks to see if Brand-Name-Drug-ABC is also cover. In this case, Brand-Name-Drug-ABC has no bioequivalent generic substitution available and Carl Adams insurance plan only covers generic drugs.

As there is no generic version available, Pharmacist Anderson is required in this example to prescribe a drug that is in the same class as Brand-Name-Drug-ABC where a generic is available. As such, Pharmacist Anderson prescribes Generic-Drug-XYZ. The prescribing of Generic-Drug-XYZ in this example is done by the pharmacist checking the drug interaction/substitution software. Pharmacist Anderson then prepares the prescription for Sildenafil and Generic-Drug-XYZ and delivers it to Carl Adams.

The record of the delivery of the prescription is sent to the insurance company so that the pharmacy can receive payment. Furthermore, the MIE record is also updated to reflect the changes of the delivered prescription. FIG. 18C illustrates the updated record of Carl Adams at the MIE after the prescriptions have been delivered.

In the examples above the most recent prescriptions are shown at the bottom of the list. However, in other examples the most recent prescriptions could be shown first and are listed for a certain duration (e.g., duration of the prescription+30 days; or if 6 renews/refills for a treatment over 30 days then 30*6+30=210 days). In other words, prescription may expire and are then not accessible by the pharmacy. Referring back to FIG. 17C, Brittany Johnson had a prescription for Vicodin® which was never delivered. In this case, the prescription was for 3 days and expired 3+30 days after it was prescribed on Oct. 1, 2013, which is November 3rd. As such, Brittany Johnson's prescription for Vicodin® was not obtained from the MIE on Jan. 10, 2014. That being said, it could have been possible for the pharmacist to request a complete history of the Brittany Johnson's record, however, the pharmacist would not be able to prescribe any expired prescriptions.

In the case of Carl Adams, as his prescription for Sildenafil has 3 refills (none of which were delivered prior the visit to the pharmacy on Jan. 10, 2014) Carl Adams's prescription is good for 30+3*30=120 days. As such, the pharmacist was able to offer a refill.

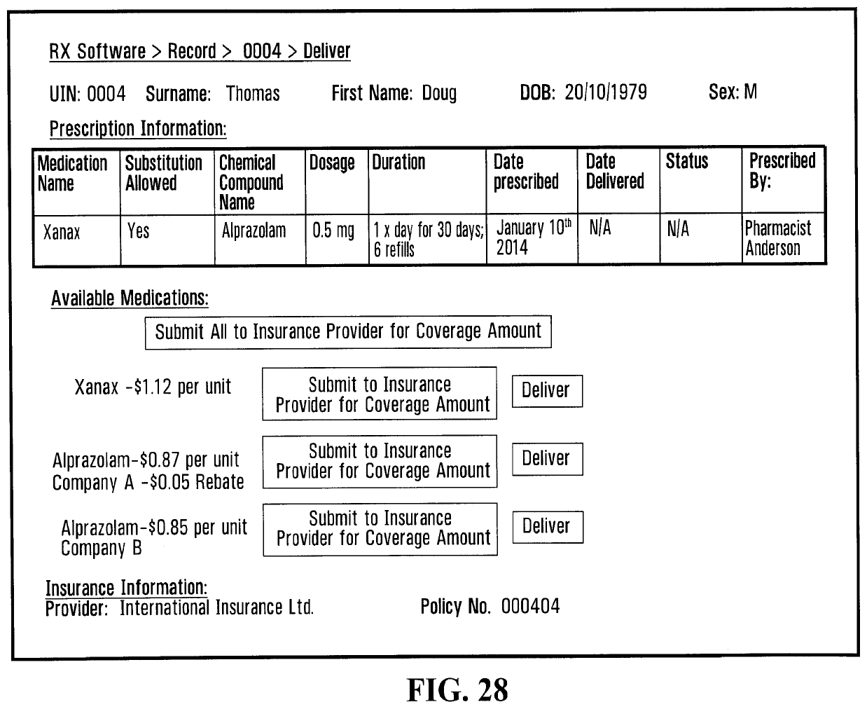

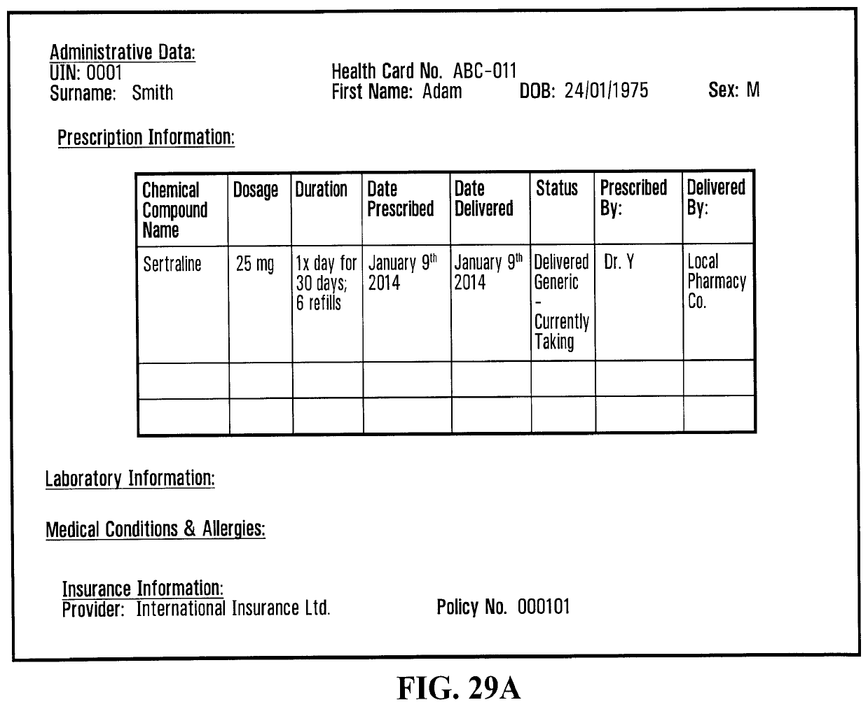

Next, Doug Thomas arrives at Global Pharmacy Inc. and is helped by Pharmacist Anderson. As in the case of the customers before, Pharmacist Anderson obtains Doug Thomas prescriptions from the MIE. Pharmacist Anderson notices that Doug Thomas prescription for Ambien® does not allow substitutions and prescribes it as is. However, Pharmacist Anderson does notice that the drug interaction/substitution software has noticed that Doug Thomas is diabetic hypoglycemic and that Escitalopram may have some unwanted side effect when taken by an individual with diabetes and that a recommended substitution would be Alprazolam (Xanax®). Pharmacist Anderson then makes the substitution for Alprazolam. In this case, as illustrated in FIG. 28, there are two different version of the generic Alprazolam available. Furthermore, Company A is offering a rebate to pharmacies and Company B is not. As such is the case, Pharmacist Anderson selects the generic version of Alprazolam from Company A as there is a rebate to the pharmacy.

The record of the delivery of the prescription is sent to the insurance company so that the pharmacy can receive payment. Furthermore, the MIE record is also updated to reflect the changes of the delivered prescription. FIG. 18D illustrates the updated record of Doug Thomas at the MIE after the prescriptions have been delivered.

Referring now back to Brittany Johnson, who has now gone to Legendary Labs Inc. to get her lab tests done. A lab technician at Legendary Labs Inc. obtains the lab request from the MIE and administers the test to Brittany Johnson. A few days later, Brittany receives an automatic phone call from the MIE indicating that here lab results are in and that she should go to a pharmacy to obtain a prescription.

As Brittany Johnson is near a different pharmacy then the one that prescribed the lab test, she enters Local Pharmacy Co. and Pharmacist Clark helps her. In this case, the lab results indicate that the offending bacteria are resistant to ciprofloxacin hydrochloride and that Brand-Name-Drug-LMN should be prescribed. As such, pharmacist Clark prescribes Brand-Name-Drug-LMN (which is covered by her insurer) and delivers the medication Brand-Name-Drug-LMN to Brittany Johnson.

The record of the deliver of the prescription is sent to the insurance company so that the pharmacy can receive payment. Furthermore, the MIE record is also updated to reflect the changes of the delivered prescription. FIG. 18B illustrates the updated record of Brittany Johnson at the MIE after the prescription has been delivered.

In the example above, the MIE stores the brand name for the drug in all cases even when substitutions are allowed. In other cases, the MIE would remove the brand name drug from each patient where substitutions are allowed and only store the chemical compound name for the prescribed drug. In other cases, the MIE would store both the brand name drug and the chemical compound name but only provide the pharmacies the brand name drug when the physician selects that no substitutions are allowed. In other cases the brand name drug name is removed prior to storing the prescription unless the doctor has indicated that no substitutions are allowed then the brand name drug name is stored.

Furthermore, in the example above, if the electronic record of a patient has already been downloaded to one of the pharmacies and updates take place to the patients file at the MIE, the update may not be automatically supplied to the pharmacy when it is entered in to the MIE. In this case, the pharmacists have to refresh the record of the patient by selecting a refresh button to download the most up-to-date record for the patient. However, alternatively, the computing entities at the pharmacies could be updated in real-time when the MIE is updated.

### EXAMPLE 2

Another specific, simplified, non-limiting example will now be provided using the connectivity of the computing entities, servers, and MIE as illustrated in FIG. 15. This example is a continuation of the features of the previous example, but where the MIE does not store the brand name drugs in the prescription records.

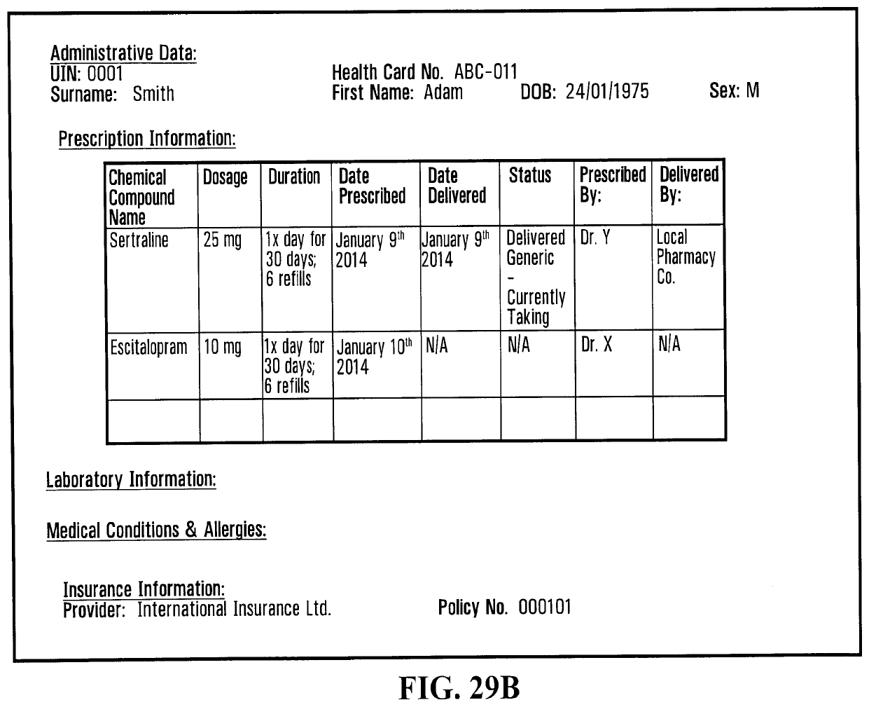

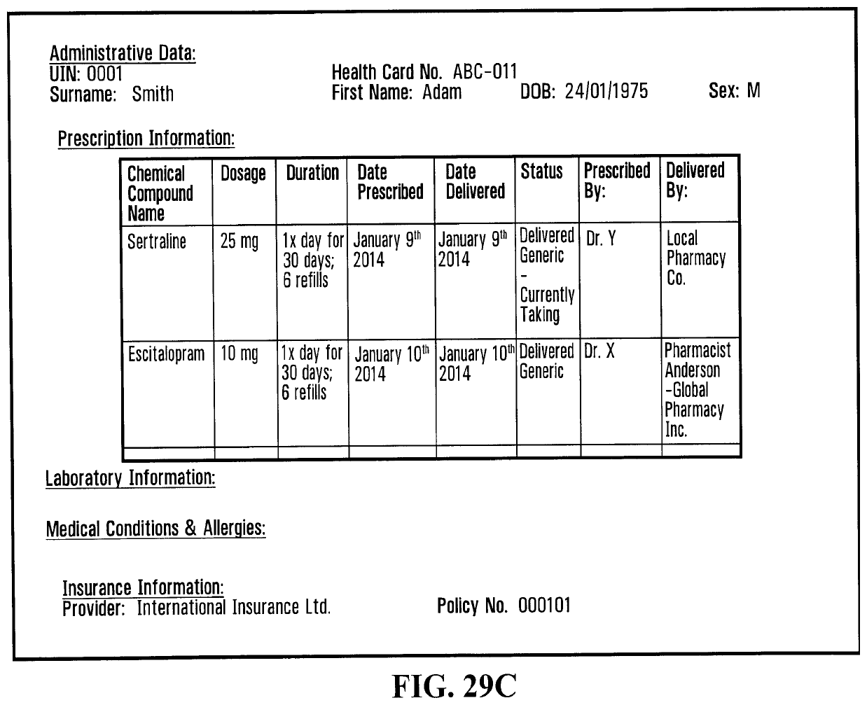

In this example, the patient Adam Smith has a medical record as shown in FIG. 29A. As illustrated, Adam Smith's medical record indicates that Dr. Y on Jan. 9, 2014 prescribed a prescription for Sertraline which was delivered the same day. As can be seen from FIG. 29A, the medical record of Adam Smith on the MIE does not contain the drug name for the prescription and only contains the chemical compound name for the drug prescribed.

Now, on January 10th, Adam Smith visits Dr. X and is prescribed a prescription for Lexapro® to help Adam with depression. In this second example, Dr. X's computer software does not have local access to a database that maps a brand name drug to the chemical compound name. As such, after Dr. X computing entity communicates with the MIE to obtain the chemical compound name for the prescribed brand name drug. That is, the chemical compound names for Lexapro® is Escitalopram. FIG. 19 illustrates a screen shot of the software running on Dr. X's computing entity which illustrates the chemical compound name obtained from the MIE after Dr. X selects Lexapro®. That is, as Dr. X selects the Lexapro® tablet size that he would like to prescribe from the list of available medications after searching for “Lexapro”, the Prescription (Rx) is loaded with both Lexapro® and Escitalopram. As such, Dr. X is able to include the chemical compound name in the prescription that he sends/registers with the MIE. FIG. 29B illustrates Adam Smith's medical record after the new prescription has been registered with the MIE. As is shown, the MIE does not store the brand name drug Lexapro® and only stores the chemical compound name Escitalopram. That is, the MIE removes the brand name drug from the prescription.

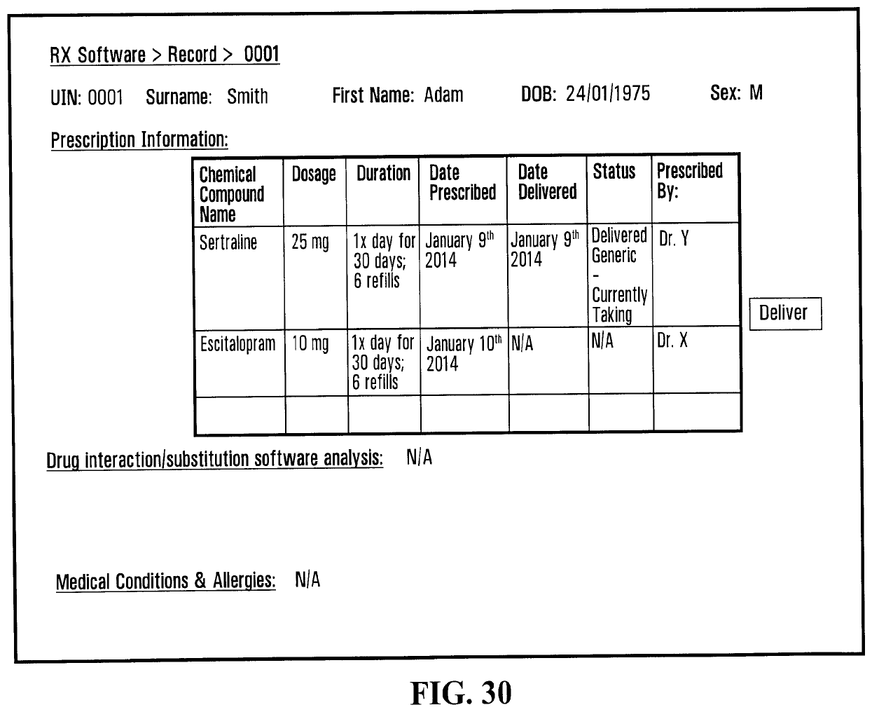

Adam Smith goes to Global Pharmacy Inc. which has one pharmacists Anderson currently working. At the beginning of his shift Anderson inserted his USB key into a port on the USB hub. Anderson then goes to workstation 1551 and login into the workstation (as was done in the previous example). Adam Smith then tells Pharmacist Anderson that he has a prescription and provides Pharmacist Anderson with his MIE's UIN (“0001”). Pharmacist Anderson then clicks on the “MIE” tab of FIG. 20, which then brings him to the screen illustrated in FIG. 21. In FIG. 21, Pharmacist Anderson using the UIN provided (“0001”) is able to located Adam Smith's medical record from the MIE and to request and downloads his undelivered prescriptions and prescription history to the pharmacy's server and computing entities. To view Adam Smith's record, Pharmacist Anderson clicks on the “Downloaded MIE Records” tab of FIG. 20. Then Pharmacist Anderson selects Adam Smith's prescription record. FIG. 30 shows the prescriptions history and available prescriptions to be delivered. Pharmacist Anderson can then deliver the prescribed prescription. Also, the MIE record is updated to reflect the changes of the delivered prescription. FIG. 29C illustrates the updated record of Adam Smith at the MIE after the prescription has been delivered.

Although in the above example, MIE does not store the brand name drug associated with the prescription. In cases where the physician prescribes a prescription where substitutions are not allowed, the MIE in these specific cases could then also store the brand name drug, which would then be provided to the pharmacy upon request of the prescription.

It is appreciated that the MIE, the servers and the computing entities discussed in this document may be implemented by one or more servers, server arrangement, or other portable or non-portable computers (including laptops, tablets, cell phones, etc.) where the one or more servers or other portable or non-portable computers include one or more databases stored in a computer readable memory and one or more computing units or processors which are programmed with software stored in a computer readable memory, which when executed executes a number of steps as set out in the software. Furthermore, the MIE, the servers and the computing entities discussed in this document may be considered different nodes in a data network (e.g., first node, second node, third node, and so forth) where each of these nodes may be remote from each other and are linked by communication paths.

It is also appreciated that the term database when referenced in this document could be a single structured table that includes at the information or it could reference to a collection of databases that could have multiple records or tables that can work jointly or independently of each other. In other words, the reference to database in this document may be to indicate the function of storage or reception of information such as patient records, summary medical records, prescription information, drug information, patient information, insurance information, etc. in one or more database, one or more tables and/or one or more records, where the databases, tables, and/or records are stored in one or more computer readable memories.

Certain additional elements that may be needed for operation of some embodiments have not been described or illustrated as they are assumed to be within the purview of those of ordinary skill in the art. Moreover, certain embodiments may be free of, may lack and/or may function without any element that is not specifically disclosed herein.

Although various embodiments and examples have been presented, this was for the purpose of describing, but not limiting, the invention. Various modifications and enhancements will become apparent to those of ordinary skill in the art and are within the scope of the invention, which is defined by the appended claims.

1.-17. (canceled)

18. Method for processing prescription information submitted to an electronic medical record system implemented in a data network including multiple nodes linked by communication paths, the multiple nodes including a first node, a second node, and a third node remote from each other, the method including:
a. receiving at a first node associated with a pharmacy prescription information associated with a patient from a server arrangement at a second node configured to receive from a third node a prescription issued by a doctor, the third node being associated with the doctor;
b. the prescription information including a data element and a second data element, the first data element being indicative of drug being dispensed to the patient, wherein the drug is a first drug and the second data element indicating whether a second drug can be substituted to the first drug;
c. forwarding to a fourth node associated with an insurance company insurance policy information associated with the patient;
d. receiving from the fourth node a third data element indicating if the second drug is covered by the insurance policy of the patient.

19. A method as defined in claim 18, including generating at the first node drug substitution information identifying at least the second drug as a possible substitute the first drug.

20. A method as defined in claim 19, including forwarding to the fourth node the drug substitution information.

21. A method as defined in claim 20, wherein the drug substitution information conveys in addition to the second drug a third drug that can be substituted to the first drug, wherein the third data element indicates if either one of the second and the third drug are covered by the insurance policy of the patient and an extent of coverage.

22. A method as defined in claim 18, including forwarding to the insurance company information about the first drug and wherein the third data element conveys one or more drugs that can be substituted to the first drug and which are covered by the policy of the patient.

23. A method as defined in claim 22, wherein the third data element conveys an extent of coverage associated with respective ones to the one or more drugs that can be substituted to the first drug.
